(19) United States
(12) Patent Application Publication
(10) Pub. No.: US 2026/0272845 A1
(43) Pub. Date: Sep. 17, 2026

(54) AQUEOUS PARTITIONING CAPSULES

(71) Applicant: KANSAS STATE UNIVERSITY RESEARCH FOUNDATION, Manhattan, KS (US)

(72) Inventors: John M. TOMICH, Manhattan, KS (US); Susan K. WHITAKER, Manhattan, KS (US); Shelby INNES, Manhattan, KS (US); Sungmin YOON, Galveston, TX (US)

(21) Appl. No.: 19/079,996

(22) Filed: Mar. 14, 2025

Related U.S. Application Data
(63) Continuation-in-part of application No. PCT/US2023/074210, filed on Sep. 14, 2023.
(60) Provisional application No. 63/406,388, filed on Sep. 14, 2022.
(60) Provisional application No. 63/485,339, filed on Feb. 16, 2023.

(51) Int. Cl. A61K 9/50; A61K 9/48; A61K 31/7105; C12N 15/88

(52) U.S. Cl. CPC A61K 9/5052; A61K 9/4808; A61K 9/5089; A61K 31/7105; C12N 15/88

(57) ABSTRACT

Aqueous partitioning peptide capsules for delivery of active agents. The capsules comprise a membrane having an exterior surface and defining a liquid-receiving interior space configured to encapsulate water-soluble active agents therein. The capsule membrane consists of a plurality of linear peptides, each peptide comprising a hydrophobic core of between 4 and 12 hydrophobic amino acids flanked by N- and C-terminal hydrophilic segments each comprising between 3 to 4 hydrophilic amino acids. Methods of making peptide capsules and methods of using the peptide capsules to deliver active agents encapsulated therein or attached thereto to a variety of organisms is described.

### CROSS-REFERENCE TO RELATED APPLICATIONS

The present application is a continuation-in-part of PCT/US2023/074210, filed Sep. 14, 2023, which claims the priority benefit of U.S. Provisional Patent Application Ser. No. 63/485,339, filed Feb. 16, 2023, and U.S. Provisional Patent Application Ser. No. 63/406,388, filed Sep. 14, 2022, each entitled AQUEOUS PARTITIONING CAPSULES, and each incorporated by reference in its entirety herein.

### SEQUENCE LISTING

The following application contains a sequence listing submitted electronically as a Standard ST.26 compliant XML file entitled “SequenceListing57230US.xml,” created on Mar. 14, 2025, as 67,122 bytes in size, the contents of which are incorporated herein.

### BACKGROUND

### Field of the Invention

The present invention relates to self-assembling peptide capsules which sequester aqueous solvents and associated solutes into their interiors for encapsulation and delivery of a wide variety of active agents.

### Description of Related Art

Nanotechnology as applied to therapeutics, known as nanomedicine, gives yield to many possibilities in healthcare. One major aspect of nanomedicine is drug delivery. Nanotechnology has improved drug delivery systems through increasing the stability of the drug or genetic material through increasing half-life, increasing the solubility for hydrophobic drugs, and aid in controlled release of the drug or genetic material.

Self-assembling nano-carriers show promise as delivery vehicles, as they overcome certain obstacles such as poor absorption that can be seen in other drug delivery classes. Compared to liposomal drug delivery systems, polymeric delivery systems have previously been shown to increase stability of the drug being delivered and aid in creating controlled release systems. Self-assembly of biomolecules is an event that occurs frequently in nature, such as lipid membranes and multi-protein complexes needed for cellular functions. The driving force for self-assembly typically involves interchain hydrogen bonds or hydrophobic interactions. Hydrogen bonding is observed in assemblies comprised of peptides that adopt β-sheet secondary structure. In the case of hydrobic interactions Van der Waals interactions predominaate. Experimentally these can be assessed by using hydrophobic solvents that compete for these interactions and lead to disassembly of the capsules. There remains a need for improved encapsulation and delivery technologies.

### SUMMARY

Here we disclose improvements in the use of linear peptides that assemble into stable hollow spheres or particles (aka capsules) for encapsulating water-soluble active ingredients in their aqueous interior. The outer surface of these capsules is hydrophilic, and preferably positively charged (said exterior surface having a Zeta potential of 0 to 40), allowing them to disperse in aqueous solutions, and fostering uptake by animal and plant tissues thereby facilitating the delivery of their payload (i.e., active ingredients) to the interior of cells. Active ingredients or targeting moieties can also be conjugated to the exterior surface of the capsules if desired. The capsules can have a particle size from about 50 to about 500 nm. Active can be selected from the group consisting of nucleic acids, fluorescent dyes, peptides, therapeutic proteins, enzymes, hydrophilic small molecule compounds and drugs, sugars, poisons, and potentiators, and combinations thereof. The capsules can also have compounds covalently or electrostatically attached to the exterior surface, such as nucleic acids, antibodies, antigens, receptor ligands, vitamins, and targeting moieties.

In one or more embodiments, the disclosure provides linear peptides comprising a hydrophobic core segment of between 4 and 12 hydrophobic amino acids and flanked by respective N- and C-terminal hydrophilic segments each comprising between 3 and 4 hydrophilic amino acids. The disclosure thus concerns aqueous compositions comprising a plurality of these linear peptides, as well as dried, shelf-stable compositions comprising a plurality of the peptides. In one or more embodiments, the peptides, when dispersed in a low-pH buffer system (preferably aqueous system) will self-assemble into capsules with a capsule membrane having a hydrophilic, and preferably positively charged, exterior surface and an interior surface defining a liquid receiving interior space (in which active agents become encapsulated).

In one or more embodiments, modified peptides can be dispersed in an aqueous buffer system over a range of pH, e.g., from pH 2 to about pH 9, and will self-assemble into capsules with a capsule membrane having a hydrophilic, and preferably positively charged, exterior surface and an interior surface defining a liquid receiving interior space (in which active agents become encapsulated). Such peptides do not require a pH shift from low pH (~2) to neutral pH (~7), thus expanding possible uses and the types of active agents that can be encapsulated.

The disclosure is also concerned with a method of forming a peptide capsule using a pH shift method. The method comprises dispersing or dissolving a plurality of the linear peptides in a low-pH buffer system, optionally comprising active agents to be encapsulated, to form a heterogeneous dispersion or solution of amphiphilic peptides (and active agent). The pH of the low-pH buffer system is preferably less than 5, more preferably less than 4, even more preferably from about 2 to about 3.5. Exemplary low-pH buffer systems include aqueous systems comprising glycine HCl, or mixtures thereof in water, preferably comprising 10 mM Glycine-HCl. The dispersion mixture is allowed to incubate, preferably at room temperature, under the low-pH conditions, for a period of time (e.g., from about 1 to about 20 minutes, preferably from about 1 to about 10 minutes, more preferably from about 1 to about 5 minutes), during which the peptides self-assemble into capsules. The pH of the mixture is then raised to a neutral pH (~7) using a neutral or alkaline buffer. It will be appreciated that suitable acidic or basic buffers (such as 15 mM Imidazole-HCl) can be used to adjust the pH to achieve the desired pH value.

The disclosure also provides a pharmaceutically acceptable composition comprising a plurality of peptide capsules, as described herein, and an active agent encapsulated in the capsules. A method of targeting delivery of an active agent to a region of a patient is also provided. The method comprises administering to a patient a peptide capsule, as described herein, which encapsulates the active agent. In one or more embodiments, methods of delivering water-soluble active agents to an organism are provided. The method comprises administering or applying a composition to the organism, such as a plant or animal, e.g., fungus, microorganism, insect, human, etc. The composition comprises a plurality of peptide capsules according to any of the embodiments described herein, in which said water-soluble active agents are encapsulated. In one or more embodiments, the organism is a plant, wherein the administering or applying comprises applying the composition to the leaves or roots of the plant, or to the soil or growth medium around the plant, optionally wherein the active agent targets a pest of the plant. In one or more embodiments, the organism is a human or non-human animal, wherein the administering or applying comprises topically applying the composition to the organism, directly administering the composition to the organism orally or via injection, or introducing the composition into an area where the organism will come into contact with the composition.

Advantageously, the capsules can further comprise a targeting moiety adducted to the exterior surface of capsule membrane for automatic selective uptake by the targeted tissue or region of interest. Advantageously, the capsules can be used to encapsulate next-gen therapeutics, such as nucleic acids, for delivery of the therapeutic payload directly into cells.

Also described herein are methods of transfecting a population of cells in vivo or in vitro with nucleic acids (e.g., mRNA). The methods generally comprise providing a plurality of peptide capsules (APCs) according to any of the embodiments described herein, in which the nucleic acids are encapsulated; and contacting the cells with the plurality of peptide capsules. In one or more embodiments, the contacting step comprises administering the peptide capsules to a subject where the capsules come into contact with the cells in vivo. In one or more embodiments, the contacting step comprises providing the population of cells in cell culture media and incubating the population of cells with the peptide capsules in the cell culture media under transfection conditions. Advantageously, the capsules are readily taken up into the cells enhancing the transfection efficiency (% of cells that have taken up the foreign nucleic acid) of the cells with the payload.

Also described herein are kits for transfecting a population of cells with nucleic acids. The kits generally comprise a plurality of lyophilized peptide capsules (APCs) according to any of the embodiments described herein, in which the nucleic acids are encapsulated; and instructions for reconstituting the lyophilized peptide capsules and incubating the population of cells with the reconstituted peptide capsules.

Also described herein are methods of forming a peptide capsule (APC) for encapsulating water-soluble active agents using the modified peptides. The methods generally comprise dispersing or dissolving a plurality of linear peptides in an aqueous solution having a pH of from about 2 to about 9, optionally comprising active agents to be encapsulated, to form a dispersion of peptides and active agent, if present, wherein the peptides each comprise a hydrophobic core comprising XLIVIIVIL (SEQ ID NO:47), where X is F or V, flanked by N- and C-terminal hydrophilic segments each comprising between 3 to 4 hydrophilic amino acids. The methods further comprise incubating the dispersion for a time period of less than 20 minutes, wherein the peptides self-assemble into peptide capsules comprising a monolayer membrane having an exterior surface and defining a liquid-receiving interior space, wherein the monolayer membrane consists of a plurality of the peptides, wherein the active agent, if present, is encapsulated in the liquid-receiving interior space of the capsule. Preferably, the hydrophobic core comprises FLIVIIVIL (SEQ ID NO:47, where X is F). Preferably, linear peptides comprise, consist essentially, or consist of KKKFLIVIIVILKKK (SEQ ID NO:56), preferably wherein the membrane consists of these linear peptides. In one aspect, the dispersing or dissolving step comprises dispersing or dissolving the plurality of linear peptides at a pH of about 7 in buffer with the active agent. In one aspect, the method further comprises drying the capsules to a dried or lyophilized powder. Advantageously, the dried or lyophilized powder can be reconstituted by mixing and suspending the powder in a neutral aqueous buffer. Additional advantages of the various embodiments of the invention are described below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows (A) a cartoon illustration of an APC and TEM images taken of APC from Ac-KKKFLIVIKKK-CONH2 (SEQ ID NO:6) at (B) pH 12, (C) pH 7, and (D) pH 2.

FIG. 2 is a graph of Circular dichroism (CD) data for APC at different pH values and in TFE.

FIG. 3 shows graphs for NTA analysis of APC formed at pH 2 and brought up to pH 7 using imidazole buffer after 5 minutes.

FIG. 4 shows graphs for NTA analysis of APC formed at pH 3 and brought up to pH 7 using imidazole buffer after 5 minutes.

FIG. 5 shows graphs for NTA analysis of APC with poly IC attached to the outside of the APC, formed at pH 2 and brought up to pH 7 using imidazole buffer after 5 minutes.

FIG. 6 shows graphs for NTA analysis of APC at pH 2 and poly IC encapsulated, formed at pH 2 and brought up to pH 7 using imidazole buffer after 5 minutes.

FIG. 7 shows a Confocal microscopy image of APC encapsulating the fluorescent dye-Rhodamine 6G. APC are roughly 2 microns in diameter.

FIG. 8 shows graphs of CD spectra of Ac-KKKFLIVIGSIIKKK-CO—NH2 (SEQ ID NO:7) APC formed and measured at pH 3.5 (A) and after adjusting pH to 7.0 (B).

FIG. 9 shows Confocal microscopy images of HEK cells with no treatment. (A) Confocal microscopy images of in vitro uptake of GFP plasmid by transfection with APC at a genetic material to peptide ratio of 1:20, attached to the outside of the APC. (B) Confocal microscopy images of in vitro uptake of GFP mRNA by transfection with APC at a genetic material to peptide ratio of 1:20, encapsulated inside the APC. (C) Characterization of these APC by DLS and Zeta measurements was also performed. (Table 12)

FIG. 10 shows Confocal microscopy images of HEK cells with no treatment. (A) Confocal microscopy images of in vitro uptake of GFP plasmid by transfection with APC at a genetic material to peptide ratio of 1:10, attached to the outside of the APC. (B) Confocal microscopy images of in vitro uptake of GFP mRNA by transfection with APC at a genetic material to peptide ratio of 1:10, encapsulated inside the APC. (C) Characterization of these APC by DLS and Zeta measurements was also performed. (Table 12)

FIG. 11 shows Confocal microscopy images of HEK cells with no treatment. (A) Confocal microscopy images of in vitro uptake of GFP plasmid by transfection with APC at a genetic material to peptide ratio of 1:5, attached to the outside of the APC. (B) Confocal microscopy images of in vitro uptake of GFP mRNA by transfection with APC at a genetic material to peptide ratio of 1:5, encapsulated inside the APC. (C) Characterization of these APC by DLS and Zeta measurements was also performed. (Table 12)

FIG. 12 shows Confocal microscopy images of HEK cells with no treatment. (A) Confocal microscopy images of in vitro uptake of GFP mRNA by transfection with APC at a genetic material to peptide ratio of 1:20, attached to the outside of the APC. (B) Confocal microscopy images of in vitro uptake of GFP mRNA by transfection with APC at a genetic material to peptide ratio of 1:20, encapsulated inside the APC. (C) Characterization of these APC by DLS and Zeta measurements was also performed. (Table 13) FIG. 13 shows Confocal microscopy images of HEK cells with no treatment. (A) Confocal microscopy images of in vitro uptake of GFP mRNA by transfection with APC at a genetic material to peptide ratio of 1:10, attached to the outside of the APC. (B) Confocal microscopy images of in vitro uptake of GFP mRNA by transfection with APC at a genetic material to peptide ratio of 1:10, encapsulated inside the APC. (C) Characterization of these APC by DLS and Zeta measurements was also performed. (Table 13)

FIG. 14 shows Confocal microscopy images of HEK cells with no treatment. (A) Confocal microscopy images of in vitro uptake of GFP mRNA by transfection with APC at a genetic material to peptide ratio of 1:5, attached to the outside of the APC. (B) Confocal microscopy images of in vitro uptake of GFP mRNA by transfection with APC at a genetic material to peptide ratio of 1:5, encapsulated inside the APC. (C) Characterization of these APC by DLS and Zeta measurements was also performed. (Table 13)

FIG. 15 shows results from RiboGreen assay of APC formed at pH 2 and incubated for 5 minutes before increasing pH to 7 using imidazole buffer. APC conditions including with encapsulated genetic material and genetic material attached to the outside of the capsules, at different ratios of genetic material to peptide.

FIG. 16 shows Confocal microscopy images of HEK cells with no treatment. (A) Confocal microscopy images of in vitro uptake of GFP mRNA by transfection with APC made at pH 2 and increased to pH 7 after 1 minute. (B) Confocal microscopy images of in vitro uptake of GFP mRNA by transfection with APC pH 2 and increased to pH 7 after 5 minutes. (C) Confocal microscopy images of in vitro uptake of GFP mRNA by transfection with APC pH 2 and increased to pH 7 after 20 minutes. (D) Characterization of these APC by DLS and Zeta measurements was also performed. (Table 14)

FIG. 17 shows Confocal microscopy images of HEK cells with no treatment. (A) Confocal microscopy images of in vitro uptake of GFP mRNA by transfection with APC made at pH 3 and increased to pH 7 after 1 minute. (B) Confocal microscopy images of in vitro uptake of GFP mRNA by transfection with APC pH 3 and increased to pH 7 after 5 minutes. (C) Confocal microscopy images of in vitro uptake of GFP mRNA by transfection with APC pH 3 and increased to pH 7 after 20 minutes. (D) Characterization of these APC by DLS and Zeta measurements was also performed. (Table 14)

FIG. 18 shows Confocal microscopy images of HEK cells with no treatment. (A) Confocal microscopy images of in vitro uptake of GFP mRNA by transfection with APC made at pH 3.5 and increased to pH 7 after 1 minute. (B) Confocal microscopy images of in vitro uptake of GFP mRNA by transfection with APC pH 3.5 and increased to pH 7 after 5 minutes. (C) Confocal microscopy images of in vitro uptake of GFP mRNA by transfection with APC pH 3.5 and increased to pH 7 after 20 minutes. (D) Characterization of these APC by DLS and Zeta measurements was also performed. (Table 14) FIG. 19 shows a graph of the CD of pH 2.0-7.0 prepared APC dissolved in increased percentages of TFE.

FIG. 20 shows Confocal microscopy images of HEK cells treated with GFP mRNA surface-bound on modified APC modified APC (using peptide SEQ ID NO:56) with different w/w ratio.

DAPI stain is used to visualize the nuclei. A) Confocal microscopy images of in vitro uptake of GFP mRNA by transfection with APC in untreated HEK293 cells. B) Confocal microscopy images of in vitro uptake of GFP mRNA by transfection with APC in cells treated with Lipofectamine 3000 (Positive Control). C) Confocal microscopy images of in vitro uptake of GFP mRNA by transfection with APC in cells treated with modified APC and GFP mRNA at a weight-to-weight (w/w) ratio of 10:1. D) Confocal microscopy images of in vitro uptake of GFP mRNA by transfection with APC in cells treated with modified APC and GFP mRNA at a weight-to-weight (w/w) ratio of 20:1. E) Confocal microscopy images of in vitro uptake of GFP mRNA by transfection with APC in cells treated with modified APC and GFP mRNA at a weight-to-weight (w/w) ratio of 50:1.

FIG. 21 shows Confocal microscopy images of HEK cells treated with GFP mRNA encapsulated in modified APC (using peptide SEQ ID NO:56) with different w/w ratio. DAPI stain is used to visualize the nuclei. A) Confocal microscopy images of in vitro uptake of GFP mRNA by transfection with APC in untreated HEK293 cells. B) Confocal microscopy images of in vitro uptake of GFP mRNA by transfection with APC in cells treated with Lipofectamine 3000 (Positive Control). C) Confocal microscopy images of in vitro uptake of GFP mRNA by transfection with APC in cells treated with modified APC and GFP mRNA at a weight-to-weight (w/w) ratio of 10:1. D) Confocal microscopy images of in vitro uptake of GFP mRNA by transfection with APC in cells treated with modified APC and GFP mRNA at a weight-to-weight (w/w) ratio of 20:1. E) Confocal microscopy images of in vitro uptake of GFP mRNA by transfection with APC in cells treated with modified APC and GFP mRNA at a weight-to-weight (w/w) ratio of 50:1.

FIG. 22 shows Confocal microscopy images of GFP mRNA expression using modified APCs with different w/w ratios for surface-bound attachment after long-term storage at room temperature. DAPI stain is used to visualize the nuclei. A) Confocal microscopy images of in vitro uptake of GFP mRNA by transfection with APC in untreated HEK293 cells. B) Confocal microscopy images of in vitro uptake of GFP mRNA by transfection with APC in cells treated with Lipofectamine 3000 (Positive Control). C) Confocal microscopy images of in vitro uptake of GFP mRNA by transfection with APC in cells treated with modified APC and GFP mRNA at a weight-to-weight (w/w) ratio of 10:1. D) Confocal microscopy images of in vitro uptake of GFP mRNA by transfection with APC in cells treated with modified APC and GFP mRNA at a weight-to-weight (w/w) ratio of 20:1. E) Confocal microscopy images of in vitro uptake of GFP mRNA by transfection with APC in cells treated with modified APC and GFP mRNA at a weight-to-weight (w/w) ratio of 50:1.

FIG. 23 shows Circular dichroism data for modified APC at different pH value and pH shift. (A) Beta-sheet secondary structure of modified peptide in TFE and random coil structure of modified APC in pH 2, 7, 8 and 9. (B) Random coil secondary structure in pH shift (pH 2 to 7, 8 and 9).

FIG. 24 shows Circular dichroism data for modified APC with nucleic acid encapsulated. (A) Random coil structure of mRNA loaded inner cargo of modified APC in pH 2, 7, 8 and 9. (B) Random coil structure in pH shifting condition (from pH 2 to 7, 8 and 9) while mRNAs are encapsulated. Modified APC:mRNA=50:1 (w/w) ratio.

FIG. 25 shows Circular dichroism data for modified APC with nucleic acid surface bound (A) Random coil secondary structure of mRNA loaded on the surface of modified APC in pH 2, 7, 8 and 9. (B) Random coil structure in pH shifting condition (from pH 2 to 7, 8 and 9) while mRNAs are encapsulated. Modified APC:mRNA=50:1 (w/w) ratio.

FIG. 26 shows Confocal microscopy images of HEK cells transfected with first generation APC's tagged with mCherry stain (A) when prepared at pH 7 and (B) when prepared at pH 3.5 and shifted to pH 7.

FIG. 27 shows Confocal microscopy images of HEK cells transfected with second generation APC's tagged with mCherry stain when prepared at pH 7, (A) Hoescht stain is used to visualize nuclei (B) mCherry is used to visualize APC transfection.

### DETAILED DESCRIPTION

Described herein are linear peptides consisting of a short hydrophobic core segment flanked by an N-terminal cationic amino acid segment and a C-terminal cationic amino acid segment, with the ability to form pH-responsive secondary structures. In some embodiments, the peptides self-assemble into water-filled nano-capsules at low pH (~3.5 or lower), which remain stable when subsequently moved after formation to physiologic pH of ~7 or greater. In some embodiments, extended peptides having strategic integration of additional valine and isoleucine residues self-assemble into water-filled nano-capsules over a range of pH values ranging from 2 to 9, including stable nano-capsules formed at pH~7. In each of the described embodiments, these capsules encapsulate water soluble active ingredients, and thus are referred to as aqueous partitioning capsules (APCs), and can deliver them to cells where they release their contents. The APCs can also be used to deliver surface-bound active agents.

The present invention is broadly concerned with compositions comprising a plurality of APCs suspended in an aqueous carrier. The APCs each comprise a peptide capsule comprising a monolayer membrane having an exterior surface and defining a liquid-receiving interior space, in which water-soluble active agents can be encapsulated. The APCs do not themselves generate any further secondary structures. They are capable of both delivery and release of their payload into a cell. Advantageously, they are able to cross the mucus layer and cell membrane, and are rapidly taken up by cells where the peptide membrane is broken down by normal intracellular processes however, they are relatively stable in solution unless an organic solvent is added to the solvent system.

The peptide sequences used to form the capsules are linear with no branch point, comprising (consisting essentially, or consisting of) a hydrophobic core segment flanked by respective N- and C-terminal cationic, hydrophilic segments. The linear peptides are generally 20 amino acid residues or less in total length, preferably from about 6 to about 20, more preferably from about 8 to about 15 residues in length. The peptides preferably have a molecular weight ranging from about 550 Da to about 2300 Da, and more preferably from about 675 Da to about 2050 Da, and even more preferably from about 800 Da to about 1800 Da. The “molecular weight” for these peptides is an average weight calculated based upon the total MW of the actual coupled amino acids present divided by the number of residues.

The hydrophobic core segment can comprise (consist essentially, or consist of) 4 to 12 amino acid residues. Amino acids used for the hydrophobic core are preferably non-polar amino acids selected from hydrophobic or very hydrophobic residues, such as leucine, isoleucine, valine, phenylalanine, tryptophan, alanine, tyrosine, and methionine. In one or more embodiments, the hydrophobic core can include up to two neutral amino acid residues selected from glycine, serine, cysteine, glutamine, and/or threonine, provided that the overall hydrophobicity of the hydrophobic segment is more hydrophobic than the hydrophilic segment, and preferably has an overall hydrophobicity of greater than 1 on the Kyte-Doolittle scale (1982). Particularly preferred hydrophobic amino acids for use in the hydrophobic segment include phenylalanine, leucine, isoleucine, and valine, with leucine being slightly less preferred among the preferred hydrophobic amino acids. If present, the neutral amino acid residues are preferably selected from glycine and/or serine. In one or more embodiments, the hydrophobic segment comprises a sequence XLIVI (SEQ ID NO: 1), XLIVIGSII (SEQ ID NO:2), XFFIVIL (SEQ ID NO:3), XLIVIGSIIVIL (SEQ ID NO: 4), or an extended sequence with valine and isoleucine additions XLIVIIVIL (SEQ ID NO:47), where X is F or V, and where the amino acid residues can be in order or in any order (scrambled, see e.g., SEQ ID NOs: 8-46 or 48-54). In one or more embodiments, the hydrophobic segment comprises a sequence X (LIVI) (SEQ ID NO:1), X (LIVI) GSII (SEQ ID NO:2), XFF (IVI) L (SEQ ID NO: 3), X (LIVI) GSIIVIL (SEQ ID NO:4), or an extended sequence with valine and isoleucine additions X (LIVI) IVIL (SEQ ID NO:47-54), where X is F or V, and where the residues in parentheses are in order or are in any order (scrambled). In one or more embodiments, the residues in parentheses are replaced with all I residues or all V residues. In one or more embodiments, any one of the residues in the following sequences can be replaced with an I or V, except for the N-terminal phenylalanine or valine: FLIVI (SEQ ID NO:1), FLIVIGSII (SEQ ID NO:2), VFFIVIL (SEQ ID NO:3), FLIVIGSIIVIL (SEQ ID NO:4), or an extended sequence with valine and isoleucine additions FLIVIIVIL (SEQ ID NO:47). In one or more embodiments, the GSII (SEQ ID NO:5) segment can include one or more substitutions to reduce the hydrophobicity of the segment, such as by replacing one or both I residues with a neutral amino acid residue.

The N- and C-terminal hydrophilic segments can comprise (consist essentially, or consist of) from 3 to 4 hydrophilic (polar) amino acid residues (each), preferably lysine, but may include arginine, histidine, aspartic acid, or glutamic acid, which also have electrically charged side chains. A particularly preferred hydrophilic segment consists of three lysine residues. The N- and C-terminal hydrophilic segments respectively can be the same or different in a given peptide sequence. The N- and C-terminal hydrophilic segments preferably have a hydrophilicity that is more hydrophilic than the hydrophobic segment.

The linear peptide sequences, preferably further comprise N-terminal acetylation and C-terminal amidation, which eliminates charge at both termini of the peptide thus allowing the peptides to assemble in either orientation (N→C or C→N). Particularly preferred peptide sequences include Ac-KKKFLIVIKKK-CONH2 (SEQ ID NO: 6), Ac-KKKFLIVIGSIIKKK-CONH2 (SEQ ID NO:7), and an extended sequence with valine and isoleucine additions Ac-KKKXLIVIIVILKKK-CONH2 (SEQ ID NO:55, were X is F or V), preferably Ac-KKKFLIVIIVILKKK-CONH2 (SEQ ID NO:56).

Surface modification of the resulting APC can also be accomplished by preemptive conjugation of functional groups and/or various moieties with the N- or C-terminal amino acid residues of the peptides, so that the modified feature will be presented on the outside of the APC surface once formed, to allow for cellular targeting. For example, the peptides can be iodinated for targeting to the thyroid gland. The term “functional moiety” is used herein to encompass functional groups, targeting moieties, and active agents that may be attached to the outer surface of the APC. Exemplary functional moieties that can be attached include fluorophores, dyes, tissue targeting moieties and ligands, antibodies, cysteine, cysteamine, biotin, biocytin, nucleic acids, polyethylene glycol (PEG), organometallic compounds, (e.g., methyl mercury), radioactive labels, conjugating chemistries, —COOH, —NH3, —SH and the like. Multiple such moieties can also be attached in a chain of sequential order from the N- or C-terminal end using aliphatic spacers to separate different moieties. Thus, the invention provides the opportunity to create multi-functionalized APC.

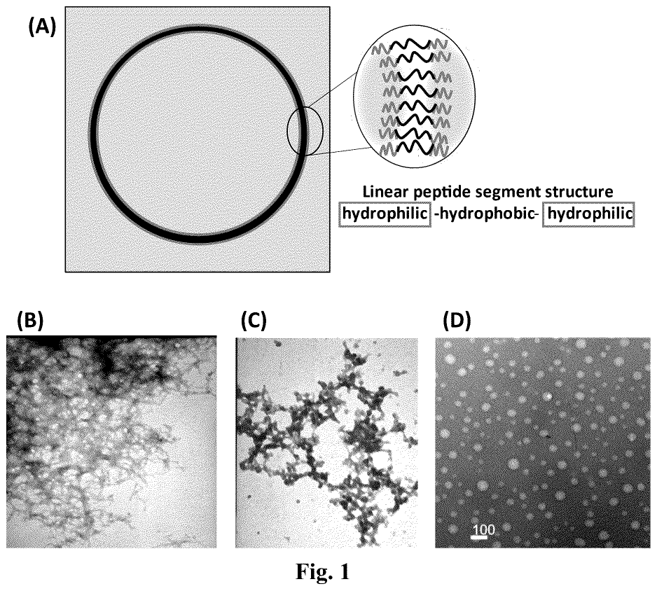

FIG. 1(A) provides an illustration of an APC according to an embodiment of the invention. In the figure, the water-soluble active agents and aqueous suspension is encapsulated or encased by a monolayer of peptide. As shown in the enlarged depiction, the squiggled lines represent the linear peptides arranged to form the peptide membrane or layer with their respective cationic N- and C-terminal hydrophilic segments facing the aqueous external environment of the suspension and the interior water-soluble active agents and aqueous suspension which have been captured by the peptides into discrete capsules. The hydrophobic core residues of each peptide forms the core of the membrane and does not directly interact with the solution once the APC forms. Importantly, the peptides themselves are not conjugated or otherwise bound to the active agents or water-soluble active agents inside the capsules, nor are the peptides covalently conjugated to each other.

Peptides can be synthesized using standard procedures. For example, solid-phase peptide synthesis can be carried out using a commercial peptide synthesizer (e.g., PS3 peptide synthesizer (Protein Technologies inc.; Tucson, AZ) or a CS Bio CS136X peptide synthesizer (CS Bio; Silicon Valley, CA)), following the manufacturers protocols. In one or more embodiments, peptides can be synthesized using Fmoc amino acids, preferably under Nitrogen gas. Once synthesis is complete, the resin can be dried under vacuum, and the peptides can be cleaved using trifluoroacetic acid (TFA), thioanisole, and/or 2% 1,2-ethanedithiol. The cleaved peptides are filtered to remove excess liquids and then precipitated using diethyl ether, followed by one or more washes to further increase the yield. The peptides are then lyophilized through suspension in deionized water. Fully dried and synthesized peptides can be stored as a solid at room temperature until use.

In one or more embodiments, when the peptides are incubated in an aqueous low-pH buffer system (i.e., pH of 3.5 or less, preferably about 2), the peptides self-assemble into capsules that encapsulate the other solutes dissolved in the solution. Thus, methods of forming the APC comprise dispersing a plurality of the linear peptides in a buffer system along with one or more active agents. Exemplary buffers systems will comprise any buffer that has a pKa of about 2.0. For example, glycine-HCl at a concentration of 10 mM is an exemplary and particularly preferred low pH buffer. For example, the buffer system can be prepared by mixing solid Glycine-HCl in distilled-deionized water to desired concentration. If necessary, dilute HCl can be added to reduce the pH of the system to the desired range (i.e., pH of 3.5 or less). Preferably, the peptides and active agents are incubated in the buffer system under low pH conditions (i.e., pH of 3.5 or less, preferably 2.5 of less, preferably about 2) for less than 20 minutes, preferably less than 10 minutes, preferably less than 5 minutes, more preferably about 1 minute or less. In one or more embodiments, the incubation is carried out under ambient conditions (i.e., temperatures of (~22° C. and normal pressure). In one or more embodiments, after mixing the components, the incubation is preferably carried out without stirring or agitation of the mixture.

In one or more embodiments, the peptides having an extended sequence with valine and isoleucine additions are incubated in an aqueous buffer system and advantageously self-assemble into capsules that encapsulate the other solutes dissolved in the solution at a pH ranging from pH 2 to pH 9. Thus, methods of forming the APCs comprise dispersing a plurality of the linear peptides in a buffer system along with one or more active agents at the optimal pH for the active agents. These modified valine and isoleucine extended peptides will assemble into APCs at pH 2. These modified valine and isoleucine extended peptides will assemble into APCs at pH 7. These modified valine and isoleucine extended peptides will assemble into APCs at pH 8. These modified valine and isoleucine extended peptides will assemble into APCs at pH 9. These modified valine and isoleucine APCs also retain their capsule structure even under a pH shift, e.g., from pH 2 to pH 7.

Advantageously, in any of the embodiments, the capsules form rapidly and minimize the amount of time that the active agents, such as nucleic acids or other (water soluble) solutes such as peptides and proteins are exposed to the low pH conditions. APC formation can be observed by the mixture changing from a suspension of peptides into capsules, which can be observed using dynamic light scattering. In applicable embodiments, the pH of the mixture can then be raised to neutral (about pH 7) using an appropriate buffer. Exemplary buffers that can be used to raise the pH include any buffer with a pKa of 1.7. Preferably the selected buffer also has no optical properties or has known properties (refractive index and viscosity) so that it can be optimized for dynamic light scattering (DLS) analysis, as the refractive index of the buffer will affect the scattering intensity, and the viscosity of the buffer will affect the diffusion coefficient of the particles. If the buffer has significant optical properties, it can interfere with the interpretation of the DLS data. For example, if the buffer absorbs light, it can reduce the amount of scattered light that is detected. If the buffer scatters light, it can create noise in the data. Thus, any suitable buffer with the appropriate pKa can be used so long as its properties have been calibrated for DLS.

Imidazole buffer at 15 mM is an exemplary and particularly preferred neutralizing buffer. If necessary, the prepared APCs can be washing using neutral buffer to remove any excess molecules used for surface conjugation, such as dyes or oligonucleotides. Otherwise, the APCs can simply be stored in the same neutralizing buffer without a need for filtering, washing, or collecting them from the suspension.

The resulting capsules are shelf-stable in neutral buffer system (about pH 7) at room temperature (~22° C.), meaning that they remain discrete capsules for extended periods of time, without agglomeration, coalescing, aggregation, or falling apart (preferably for at least 3 months, more preferably at least 6 months, even more preferably at least 12 months). The capsules generally have a maximum surface-to-surface dimension (e.g., the diameter of a substantially spherical capsule) of less than 500 nm, preferably from about 50 to about 500 nm, preferably about 100 to about 300 nm. For ease of reference, the terms “diameter” or “particle size” are used interchangeably herein to refer to the maximum surface-to-surface dimension of each capsule. Moreover, since the methods of the invention yield suspensions of a plurality of capsule, the “particle size” referenced herein may refer to the average (mathematical mean) diameter of the entire population of capsules in the suspension. Exemplary neutral buffer systems that can be used for shelf-stable storage include imidazole buffer at 15 mM. In one or more embodiments, the storage buffer further includes histidine or buffers substantially free of any sulfate or phosphate conjugate acids, for improved stability over time. In one or more embodiments, the storage buffer can include up to 20% DMSO for cryopreservation. Advantageously, the APCs are biodegradable, meaning that they remain intact and will protect nucleic acids from degradation, but will themselves eventually break down over time in an environmentally friendly manner.

A wide variety of active agents can be delivered using the APCs including water-soluble active agents that can be encapsulated in the APCs as well as other agents that can be conjugated to the outside of the APCs. For example, the water-filled capsules are suitable for delivering nucleic acids (DNA, plasmid DNA, RNA, mRNA, siRNA, microRNA, dsRNA and the like) for cellular delivery, as well as other active agents, such as enzymes, peptides, hydrophilic small molecule compounds and drugs, sugars, poisons, and potentiators, as well as detectable labels, such as dyes or fluorescent markers, etc. The capsules are also suitable to deliver a variety of materials covalently or electrostatically attached to its outer surface, such as antibodies, antigens, receptor ligands, vitamins, and other targeting moieties.

These capsules can be used to deliver water-soluble active ingredients to plants, non-human animals (including insects, fish, birds, mammals, reptiles, etc.), and humans both in vitro, ex vivo, and in vivo. Thus, also contemplated herein are methods for delivering active agents to a plant, animal, or human. The methods comprise administering a plurality of peptide capsules containing an active ingredient to the plant, non-human animal, or human. This can include directly applying or administering the peptide capsules, or providing the peptide capsules to the vicinity of the target plant, non-human animal, or human. For example, the peptide capsules may be applied directly to a plant leaf or root system, and/or may be applied to the soil around the roots. Likewise, the peptide capsules can be directly administered topically, orally, or via injection into the non-human animal or human, or may be introduced indirectly, for example, into aquaculture/water system in which the animal resides (such as fish, crustaceans, etc.), or in a location where the non-human animal may come into contact with it (e.g., near a beehive to treat bees or bee pests, etc.). Advantageously, the peptide capsules are taken up intracellularly by the organism after administering or applying thereto, such that the active agent is delivered intracellularly. Further, the peptide capsule membrane are configured to be broken down by endogenous intracellular mechanisms in the organism to release the active agent. The peptide capsules may be incorporated into a suitable pharmaceutical, horticultural, or veterinary composition, including a suitable carrier, diluent, excipient, or vehicle for administration. Exemplary carriers and vehicles include, without limitation, sugars, polysaccharides, and glycerol.

For human or animal use, suitable carriers will be pharmaceutically acceptable. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable carrier would be selected to minimize any degradation of the compound or other agents and to minimize any adverse side effects in the subject. Pharmaceutically-acceptable ingredients include those acceptable for veterinary use as well as human pharmaceutical use, and will depend on the route of administration. For example, compositions suitable for administration via injection are typically solutions in sterile isotonic aqueous buffer, such as phosphate buffered saline (PBS), and the like. Exemplary carriers include aqueous solutions such as mono and disaccharides, and the like.

The composition can comprise a therapeutically effective amount of the peptide capsules (containing the active ingredient) dispersed in the carrier. As used herein, a “therapeutically effective” amount refers to the amount that will elicit the biological or medical response of a tissue, system, or subject that is being sought by a researcher or clinician, such as to elicit some desired therapeutic or prophylactic effect as against the disease or condition. It will be appreciated that in the case of delivery of pesticidal or herbicidal active agents, the desired effect is inhibited growth of, damage to, and/or death of the target. One of skill in the art recognizes that an amount may be considered therapeutically “effective” even if the disease, condition, or pest is not totally eradicated or prevented, but it or its symptoms and/or effects are improved or alleviated partially in the subject.

Other ingredients may be included in the composition, such as adjuvants, other active agents, preservatives, buffering agents (e.g., histidine), salts, and other pharmaceutically-acceptable ingredients. The term “adjuvant” is used herein to refer to substances that have immune-potentiating effects and are added to or co-formulated in a therapeutic composition in order to enhance, elicit, and/or modulate the innate, humoral, and/or cell-mediated immune response against the active ingredients. The compositions have a stability of 1-2 weeks once the APCs are formed. Advantageously, however, the peptides, particularly in lyophilized form are extremely stable and have a shelf-stability of at least about 90 days. They can be provided in kits to prepare suitable APC formulations on-site before administration.

In some embodiments, the peptides, APCs, or compositions can be provided in unit dosage form in a suitable container. The term “unit dosage form” refers to a physically discrete unit suitable as a unitary dosage for plant, non-human animal, or human use. Each unit dosage form may contain a predetermined amount of peptide, pre-formed APCs, or active agents, in a suitable carrier calculated to produce a desired effect. In other embodiments, the peptides can be provided separate e.g., in their own vial, ampule, sachet, or other suitable container from the active ingredient, for on-site mixing with a vehicle or carrier and APCs formation, before administration to the subject plant, non-human animal, or human. A kit comprising the peptides, pre-formed APCs, and/or active agents is also disclosed herein. The kit further comprises instructions for preparing the APCs (if necessary) and administering the composition to the subject.

It will be appreciated that therapeutic and prophylactic methods described herein are applicable to humans as well as any suitable non-human animal, including, without limitation, dogs, cats, and other pets, as well as, rodents, primates, horses, cattle, pigs, fish, birds, etc., including targeting pathogens and pests (e.g., fungus, insect, or bacteria) in human and non-human animal systems or otherwise delivering nucleic acids or other active agents to human and non-human animal tissues. This platform technology is also useful in plants, such as for targeting pathogens and pests in plant systems (e.g., fungus, insect, or bacteria) or otherwise delivering nucleic acids or other active agents to plant tissues. The methods can be also applied for clinical research and/or study.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Abstract

The Aqueous Partitioning Capsules (APC) forming peptides have a primary sequence of Ac-KKKFLIVIKKK-CONH2 (SEQ ID NO:6) and Ac-KKKFLIVIGSIIKKK-CONH2 (SEQ ID NO: 7). Transmission electron microscopy analysis of the assemblies formed by Ac-KKKFLIVIKKK-CONH2 (SEQ ID NO:6) at pH 2 revealed spherically shaped images. Further studies on the pH 2 assemblies indicate that while low pH is essential for self-assembly, they remained stable when moved to neutral pH. The APC are efficient in delivering GFP encoding mRNA and plasmids human embryonic kidney (HEK) cells. The stability of the APC encapsulating GFP mRNA and GFP plasmid were studied over time. RiboGreen assays were utilized to confirm the location of the genetic material on either the outside or the inside of the APC. Preliminary studies of the APC encapsulation abilities, stability over time, and delivery of GFP mRNA and plasmid to HEK cells are presented.

When the peptides are dispersed in water at low pH (~2-3.5), the peptides form nanosized spherical structures, juxtaposing the pH 7 and pH 12 images showing aggregates and nanofibrils, respectively, as shown in FIG. 1. One skilled in the art would not anticipate or predict the formation of water-filled capsules at low pH since the net charge on the entire molecule does not change upon lowering the pH from neutral~7 to low pH. The exact mechanism for the low pH assembly remains an area of investigation. Here we report the unexpected ability of APC to be formed under controlled conditions for encapsulation of various types of molecules, including dye, mRNA, and dsDNA. At low pH the nucleic acids lose their negative charge, as the phosphates in the backbone become protonated. It is not until the capsules are placed in a neutral pH environment that the charges return to the nucleic acids. Along with encapsulation, attachment to the outside of the APC (formed at low pH and then raised to neutral pH) was explored, due to the large number of lysine residues in the APC primary sequence, that hold a positive charge at both acidic and neutral pH. The negatively charged genetic material is proposed to electrostatically attach to these positively charged lysine residues.

### Materials and Methods

Solid-phase peptide synthesis—The peptides, Ac-KKKFLIVIKKK-CONH2 (SEQ ID NO: 6) and Ac-KKKFLIVIGSIIKKK-CONH2 (SEQ ID NO:7) were synthesized using solid-phase peptide synthesis on a PS3 peptide synthesizer (Protein Technologies inc.; Tucson, AZ). Peptide synthesis was completed on a 0.1 mmol scale using Fmoc (fluorenylmethyloxycarbonyl). The Fmoc amino acids were obtained from AnaSpec, Inc. (San Jose, CA). The synthesis was carried out using the automated PS3 peptide synthesizer (Company and City). The peptides were cleaved at room temperature for 90 minutes, using 92% trifluoroacetic acid (TFA), 5% thioanisole, and 2% 1,2-ethanedithiol, to yield TFA-salt forms of the peptides. The liquid was removed after the cleavage and poured into ice cold diethyl ether. Three more consecutive washes using diethyl ether were done to precipitate the peptide. Both peptides were lyophilized for storage and subsequently suspended using deionized water for the following studies.

Preparation of APC—Peptides were dissolved in 2,2,2-Trifluoroethanol (TFE). The concentration of peptide was determined by measuring the absorbance of phenylalanine at a wavelength of 258 nm, on a CARY 50 Bio UV-visible spectrometer (Agilent Technologies, Santa Clara, CA). Final concentrations of 1 mM APC were prepared by adding peptide dissolved in TFE into an Eppendorf tube. Then, the solvent was removed using a vacuum and rotor. After drying, the APC peptides were rehydrated at different pH values ranging from 2 to 4. The APC were incubated at room temperature for various times before increasing the pH to 7. The sizes of the peptide nanoparticles (capsules) were measured.

Dynamic light scattering and Zeta potential characterization—APC capsules were prepared at a concentration of 1 mM as previously described (Preparation of APC and Preparation of mRNA- or dsDNA-APC's nanoparticles). The particle size and Zeta potentials for each of the samples was analyzed using a Zetasizer Nano ZS (Malvern Instruments Ltd, Westborough, MA).

NTA studies—Nanoparticle tracking analysis was performed using a NanoSight LM 14 (Malvern Panalytical, UK). A single chamber was connected to a 405 nm laser. The type of camera used was a Hamamatsu Photonics K. K. CMOS camera Model #C11440-50B. The samples were injected into the single chamber using sterile syringes (BD Discardit II, New Jersey, USA). Videos were taken of the samples at 25.0 FPS over a period of 60 seconds. The temperature was kept consistent at 25.0° C. APC capsules were prepared at a concentration of 1 mg/mL as previously described (Preparation of APC) at both a pH of 2 and a pH of 3. Samples containing poly IC, dsDNA, were prepared at a ratio of 1:10 genetic material to peptide as previously described (Preparation of mRNA- or dsDNA-APC's nanoparticles). NanoSight NTA 3.3 was the software used to analyze the captured images to calculate the number and size of the APC nanoparticles in solution.

Circular dichroism—Circular dichroism (CD) spectrum data was collected using a Jasco-815 CD spectrophotometer (Jasco Analytical Instruments, Easton, MD). The type of cuvette used to collect data was a 1 mm path length quartz cuvette. The wavelength range scanned was 260 nm to 190 nm, using 50 nm/min as the scan rate. 1 nm step intervals were used during the scans. A total of five scans were recorded and averaged for each sample. Ellipticity was measured using millidegrees as the units. Data was corrected based on the solvent used, in these CD measurements either TFE, water, or imidazole buffers were used as solvents. A Savitsky-Golay filter was used to smooth the spectrum. This analysis software was provided by the manufacturer of the CD spectrophotometer.

APC dye encapsulation—Two samples of APC at 1 mM concentration were prepared at pH 2 and pH 2, increased to 7, as previously described (Preparation of APC). 5 μL of the rhodamine 6G stock solution was added to the 1.0 mL of APC, to give a final concentration of rhodamine 6G of 100 μM. Samples were washed with water three times and spun at 3000 RCF for 3 minutes at 25° C. using a Labnet Prism® tabletop centrifuge (Labnet, Edison, NJ). The flow through after the third was clear. The fourth was with 200 μL of 200 mM sodium trifluoroacetic acid (Na*TFA) at 3000 RCF for 3 minutes at 25° C. A final fifth spin was for 1 minute at 3000 RCF to quickly remove any remaining unencapsulated rhodamine 6G dye.

Samples were analyzed on a CARY Eclipse fluorescence spectrophotometer (Agilent Technologies, Santa Clara, CA). Samples of APC with encapsulated rhodamine 6G dye were placed onto a glass slide and allowed to dry. The slide was analyzed using a confocal LSM 700 laser-scanning microscope (Carl Ziess, Gottingen, Germany). Images taken on the confocal microscope were analyzed using ZEN 3.5 (blue edition).

Longevity studies—APC capsules were prepared as previously described (Preparation of APC and Preparation of mRNA- or dsDNA-APC's nanoparticles) and stored at either room temperature (22° C.) or at 4° C. The particles sizes and Zeta potentials of each sample were analyzed over time using a Zetasizer Nano ZS (Dynamic light scattering and Zeta potential characterization).

Preparation of mRNA—or dsDNA-APC's nanoparticles-Different ratios of genetic material to APC were made by first drying the APC peptides using a vacuum and rotor. The desired amount of APC peptides in the final ratio was taken from a stock solution of 1 mM APC in TFE and placed into a microcentrifuge tube. The microcentrifuge tube was placed into a rotor with a vacuum to dry the APC peptides. For encapsulation of the genetic material, 200 ng of the genetic material was added to 20 μL of pH 2 glycine-hydrochloric acid (HCl) buffer. The 20 μL of pH 2 buffer with the genetic material was used to rehydrate the APC peptides and form the APC. For attachment of the genetic material to the outside of the APC, 20 μL of the pH 2 glycine-HCl buffer was used to rehydrate the APC peptides. The APC peptide solution was allowed to incubate for 5 minutes at room temperature in the pH 2 buffer, before adding 200 ng of genetic material.

APC GFP plasmid transfection—Human embryonic kidney (HEK) cells were grown in an incubator at 37° C. with 5% CO2. The HEK cells were grown in 4.5 g/L glucose Dulbecco's Modified Eagle medium (DMEM) with penicillin-streptomycin and fetal bovine serum (FBS). Cells were grown in an 8-well cell tray to prepare for transfection.

Different ratios of APC to plasmid were tested during the transfection, along with testing both attachment to the outside of the APC and encapsulation within the APC. The conditions tested during this plasmid transfection included cells only, lipofectamine (as a positive control), 1:5 outside, 1:5 inside, 1:10 outside, 1:10 inside, 1:20 outside, and 1:20 inside. The ratios always included 200 ng of genetic material. The amount of peptide was 1 μg in the 1:5 ratios, 2 μg in the 1:10 ratios, and 4 μg in the 1:20 ratios. Each amount of peptide was dried using a rotor and vacuum. After drying the APC, the outside conditions were rehydrated using 20 μL pH 2 solution immediately after finishing drying. The tubes were allowed to incubate for 5 minutes at room temperature before adding 200 ng of GFP plasmid. For the inside conditions, 200 ng GFP plasmid for each condition was added to the 20 μL of pH 2 buffer. The GFP plasmid and pH 2 buffer were used to rehydrate the APC. All tubes were incubated at room temperature for 5 minutes before bringing the pH up to 7 using 80 μL imidazole buffer.

HEK cell media was changed immediately before transfection. Conditions were added to separate wells in the cell tray. The transfection was allowed to incubate for 44 hours. The transfection was terminated using perfluoroalkoxy alkanes (PFA). After termination of the transfection, the media was replaced with phosphate buffered saline (PBS). To permeabilize the cell membranes, Triton X was added to the wells and allowed to sit for approximately 1 hour. The cells were washed with PBS. The cell tray was then allowed to completely dry before adding SlowFade™ Diamond Antifade Mountant with DAPI. A glass coverslip was placed on top of the cell tray and sealed using clear nail polish. The cell tray was covered in aluminum foil and stored at room temperature before analyzing on a confocal microscope. Images taken on the confocal microscope were analyzed using ZEN 3.5 (blue edition).

APC GFP mRNA transfection—Human embryonic kidney (HEK) cells were grown in an incubator at 37° C. with 5% CO2. The HEK cells were grown in 4.5 g/L glucose Dulbecco's Modified Eagle medium (DMEM) with penicillin-streptomycin and fetal bovine serum (FBS). Cells were grown in an 8-well cell tray to prepare for transfection.

APC at ratios of 1:5, 1:10, and 1:20 were prepared as previously described (APC GFP plasmid transfection). APC with GFP mRNA encapsulated and attached to the outside of the capsule were prepared for each ratio.

The outside conditions were rehydrated using 20 μL pH 2 solution immediately after finishing drying. The tubes were allowed to incubate for 5 minutes at room temperature before adding 200 ng of GFP mRNA. For the inside conditions, 200 ng GFP mRNA for each condition was added to the 20 μL of pH 2 buffer. The GFP mRNA and pH 2 buffer were used to rehydrate the APC. All tubes were incubated at room temperature for 5 minutes before bringing the pH up to 7 using 80 μL imidazole buffer.

HEK cell media was changed immediately before transfection. Conditions were added to separate wells in the cell tray. The transfection was allowed to incubate for 44 hours. The transfection was terminated using perfluoroalkoxy alkanes (PFA). After termination of the transfection, the media was replaced with phosphate buffered saline (PBS). To permeabilize the cell membranes, Triton X was added to the wells and allowed to sit for approximately 1 hour. Cells were washed with PBS. The cell tray was then allowed to completely dry before adding SlowFade™ Diamond Antifade Mountant with DAPI. A glass coverslip was placed on top of the cell tray and sealed using clear nail polish. The cell tray was covered in aluminum foil and stored at room temperature before analyzing on a confocal microscope. Images taken on the confocal microscope were analyzed using ZEN 3.5 (blue edition).

RiboGreen assay—The RiboGreen assay was used since the RiboGreen reagent is a fluorescent dye that can detect genetic material. The RiboGreen reagent should detect genetic material attached to the outside of the APC capsules, but not genetic material encapsulated in the APC capsules. The ratio of genetic material to peptide used in this assay were 1:1, 5:1, and 10:1. The APC nanoparticles were prepared at these ratios as previously described (Preparation of APC and Preparation of mRNA- or dsDNA-APC's nanoparticles). The assay was set up on a 96 well plate. Each well with APC nanoparticles received 0.5 μL RiboGreen agent in 100 μL of TE buffer. After adding the reagent, the fluorescence was determined using a plate reader.

APC GFP mRNA transfection efficacy study—HEK cells were prepared as previously described (APC GFP mRNA transfection). APC were each prepared at a ratio of 1:10 with the GFP mRNA encapsulated. Each sample was rehydrated with 200 ng GFP mRNA and 20 μL pH 2, pH 3, or pH 3.5 solution. Each sample incubated at room temperature for either 1 minute, 5 minutes, or 20 minutes before increasing the pH to 7 using 80 μL imidazole buffer. The samples were added to HEK cells which incubated for 44 hours before terminating the transfection using PFA. Cells were prepared for confocal microscopy as previously described (APC GFP mRNA transfection).

### Characterization of APC

Our studies initially focused on the shorter peptide (SEQ ID NO:6, KKKFLIVIKKK-CONH₂) sequence. The sizes of the KKKFLIVIKKK-CONH2 (SEQ ID NO:6) APC, formed at and or transferred to three pH values (pH 2, 7 and 12) were analyzed using DLS. The size of APC were smallest at pH 2, showing an average diameter of 239.1, with a standard deviation of 28.40 nm over the course of three trials (Table 1).

TABLE 1
Properties of KKKFLIVIKKK-CONH₂ (SEQ ID NO: 6) APC at different pH values and
in TFE. Each sample was read three times and the results are the average of those runs,
with the standard deviation included in the table for the size and hydrodynamic diameter.

| | Hydrodynamic | | | Zeta Potential |
|---|---|---|---|---|
| Sample | Diameter (nm) | Polydispersity | Size (nm) | (mV) |
| TFE | 1361 ± 226 | 34.0% | 686.6 ± 209 | n/a |
| pH = 2 | 239.1 ± 28.40 | 20.70% | 155.8 ± 32.7 | 6.4 |
| pH = 7 | 664.3 ± 448 | 29.2% | 243.5 ± 56.9 | 1.2 |
| pH = 12 | 3406 ± 1832 | 29.0% | 2047.5 ± 1283 | −3.9 |
| pH = 2 to 7 | 340.5 ± 238.4 | 26.1% | 175.6 ± 99.6 | 9.3 |
| pH = 2 to 12 | 7229.3 ± 4818 | 28.0% | 3015 ± 2148 | −0.6 |

The APC were largest at pH 12, being 3406 nm, with a standard deviation of 1832 over the course of three trials (Table 1). At pH 7, the size of the APC was 664.3 nm, however, when initially making the APC at a pH 2 and increasing the pH to 7, the size was measured, 340.5 nm, in close in size to the pH 2 assemblies. These results show that the APC can be initially made at a pH 2 then raised to a pH 7 and maintain similar size and net positive charge. This is consistent with the CD measurements, which showed forming the capsules at a low pH and later raising the pH, does not change the random coil secondary structure formed at a low pH. The Zeta potentials at each pH value all remained positive. The positive Zeta potentials indicate that a positive surface charge may be caused by the positively charged lysine residues being exposed on the surface of the APC capsules.

The first experiments characterizing the APC focused on DLS, and Zeta potential measurements and CD. Dynamic light scattering was used to determine hydrodynamic diameter, polydispersity, and surface charge (Zeta potential). Circular dichroism was used to determine the secondary structure of APC at different pH values. At a pH of 2, the APC are shown (FIG. 1) to form a random coil secondary structure. At a pH of 7 and 12, the APC are shown to form β-sheets. In TFE, the APC peptides are shown to form α-helices. Of note is the fact that when the peptide is allowed to assemble at pH 2, increasing the pH to 7 does not induce the β-sheet secondary structure, but instead remains as a random coil secondary structure (FIG. 1). This observation implies that once self-assembled at low pH, the secondary structure becomes locked and resistant to change when moved to elevated pH values.

The results for the KKKFLIVIKKK-CONH2 (SEQ ID NO:6) APC formed for 1, 3, 4, and 5 min were recorded (Table 2). Even at the earliest timepoint, of 1 minute, self-assembly appears complete. The polydispersity values look excellent with the capsule sizes just under 100 nm. The sizes for the 4 and 5 min incubation periods are an artifact, due to small sample size. The Zeta potentials of each sample all were shown to be positive values, again indicating that the surface charge is positive due to the lysine residues exposed to the surface.

TABLE 2
Dynamic light scattering and Zeta potential values at pH 2.0 for
APC formed over different times prior to raising pH to 7.0.

| Time | Hydrodynamic | Poly- | Size | Zeta Potential |
|---|---|---|---|---|
| (minutes) | Diameter (nm) | dispersity | (nm) | (mV) |
| 1 | 423.1 | 28.80% | 74.19 | 0.3 |
| 3 | 417.7 | 24.40% | 91.42 | 0.6 |
| 4 | 403.7 | 26.30% | 0.41 | 3.6 |
| 5 | 11920 | 43.20% | 1.02 | 2.0 |

For the next study APC formation was followed for two time periods, using a wider pH range of 2.00-4.56 (Tables 3 and 4). The two incubation times gave similarly sized APC for pH values up to pH 3.5. Above pH 3.5, the hydrodynamic sizes started to increase, a characteristic of materials that are beginning to aggregate. For the 5-minute incubation samples, sizes of the capsules remained around 100 nm until pH 3.51, in which they showed signs of aggregation due to an extreme drop in size to 13 nm (Table 3). The Zeta potentials remained positive through each of the pH values, with the highest being 14.3 mV at pH 2 and the lowest being 5.9 mV at pH 3.51. For the 20-minute incubation samples, sizes of the capsules remained around 100 nm until pH 3.51, in which they showed signs of aggregation due to an extreme drop in size to 23 nm (Table 4). This drop in size indicates that aggregation may have begun to occur at this pH. The Zeta potentials remained positive through each of the pH values, with the highest being 14.4 mV at pH 2 and the lowest being 5.5 mV at pH 3.90. Overall, at each incubation time, the APC appear to show signs of aggregation at pH greater than 3.51. The Zeta potentials of the APC have the greatest positive values at pH 2 and decrease as the pH increases. However, even at pH 4.56, the Zeta potentials remain positive, indicating that the lysine residues on the outside of the capsules are protonated.

TABLE 3
Characteristics of APC at different pH values and with an
incubation time of 5 minutes before increasing the pH to 7.

| | Hydrodynamic | Poly- | Size | Zeta Potential |
|---|---|---|---|---|
| pH | Diameter (nm) | dispersity | (nm) | (mV) |
| 2.00 | 320.2 | 25.00% | 108.72 | 14.3 |
| 2.49 | 336.1 | 26.80% | 119.75 | 8.2 |
| 3.00 | 304.9 | 21.50% | 116.64 | 15.9 |
| 3.30 | 359.3 | 28.90% | 99.80 | 10.8 |
| 3.51 | 308.0 | 24.70% | 13.36 | 5.9 |
| 3.90 | 785.1 | 23.10% | 65.40 | 10.5 |
| 4.56 | 376.3 | 26.80% | 107.98 | 7.5 |

TABLE 4
Characteristics of APC at different pH values and with an
incubation time of 20 minutes before increasing the pH to 7.

| | Hydrodynamic | Poly- | Size | Zeta Potential |
|---|---|---|---|---|
| pH | Diameter (nm) | dispersity | (nm) | (mV) |
| 2.00 | 518.4 | 27.00% | 149.3 | 14.4 |
| 2.49 | 368.6 | 23.90% | 49.68 | 7.70 |
| 3.00 | 452.3 | 25.40% | 20.16 | 8.30 |
| 3.30 | 432.2 | 30.10% | 129.4 | 7.00 |
| 3.51 | 273.3 | 26.60% | 23.27 | 11.6 |
| 3.90 | 601.0 | 30.60% | 123.4 | 5.50 |
| 4.56 | 674.3 | 35.20% | 159.14 | 8.50 |

### Nanoparticle Tracking Analysis

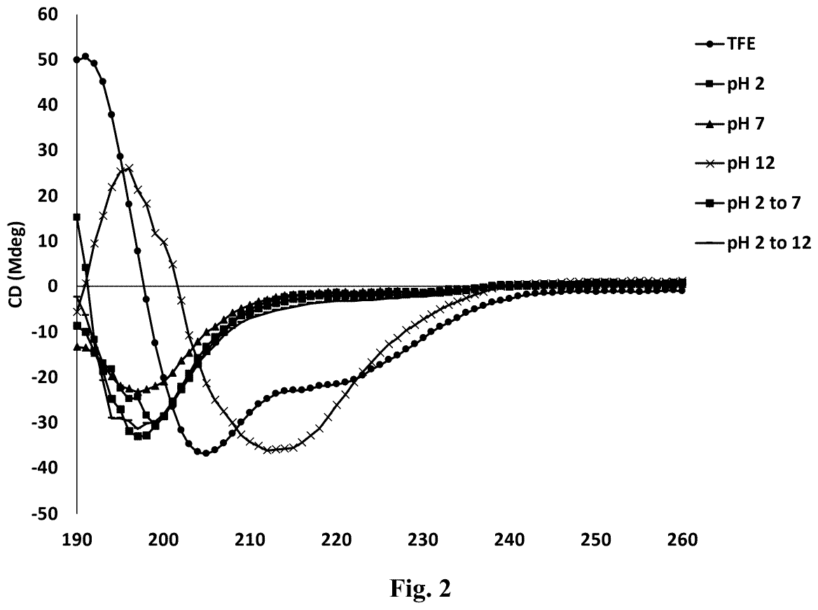

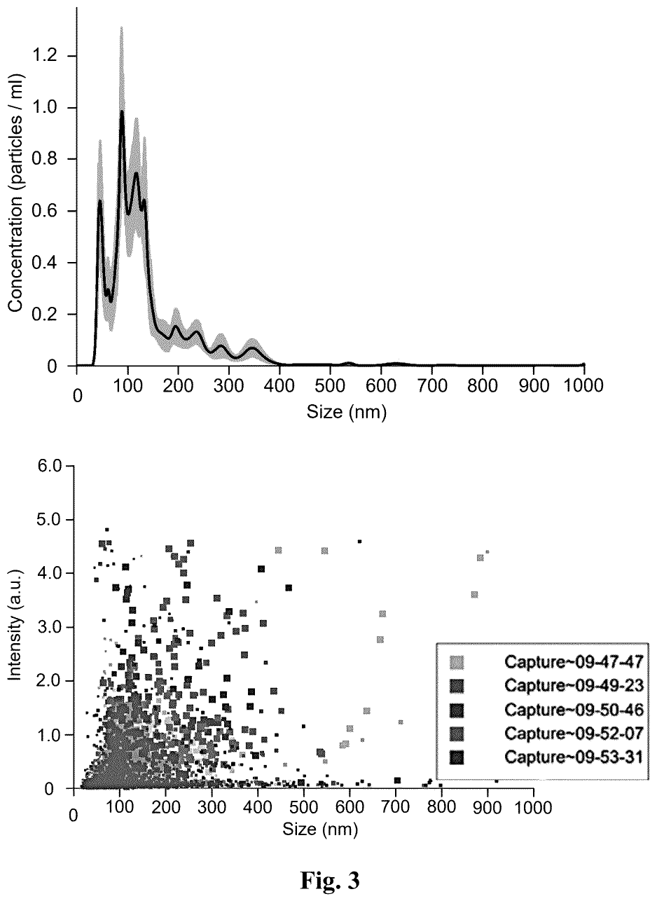

To better examine the size and distribution of APC capsules, Nanoparticle Tracking Analysis (NTA) was performed to show the size distribution for APC formed at pH 2 and raised to pH 7 after 5 minutes (FIG. 2). APC formed at a pH of 3 were analyzed as well, showing similar results to the APC formed at a pH 2 (FIG. 3). The analysis of these APC shows that most of the particles vary in diameter from 50-200 nm, with very few particles larger than 300 nm. The mean size for the APC formed at pH 2 was 139.1 nm with a standard error of 8.9 nm. The mean size for the APC formed at pH 3 was 240.1 nm with a standard error of 104.0 nm. These results are consistent with the results produced by running the samples on DLS (Tables 1 and 3). Both the NTA and DLS showed that the APC formed at pH 2 and raised to pH 7 are approximately 100-200 nm in size. Similarly, the APC formed at pH 3 showed similar results on both NTA and DLS, showing that the size ranges from 200-300 nm in size.

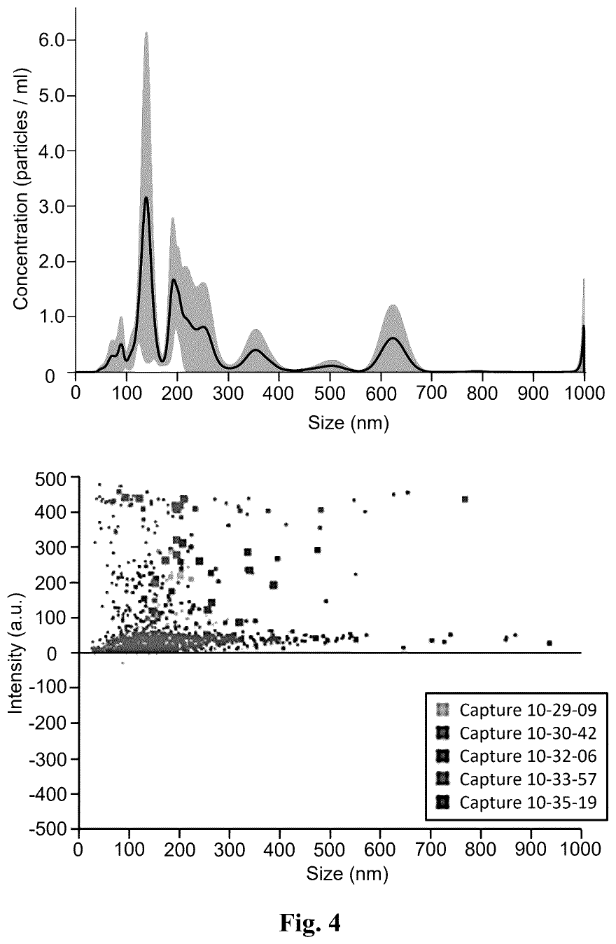

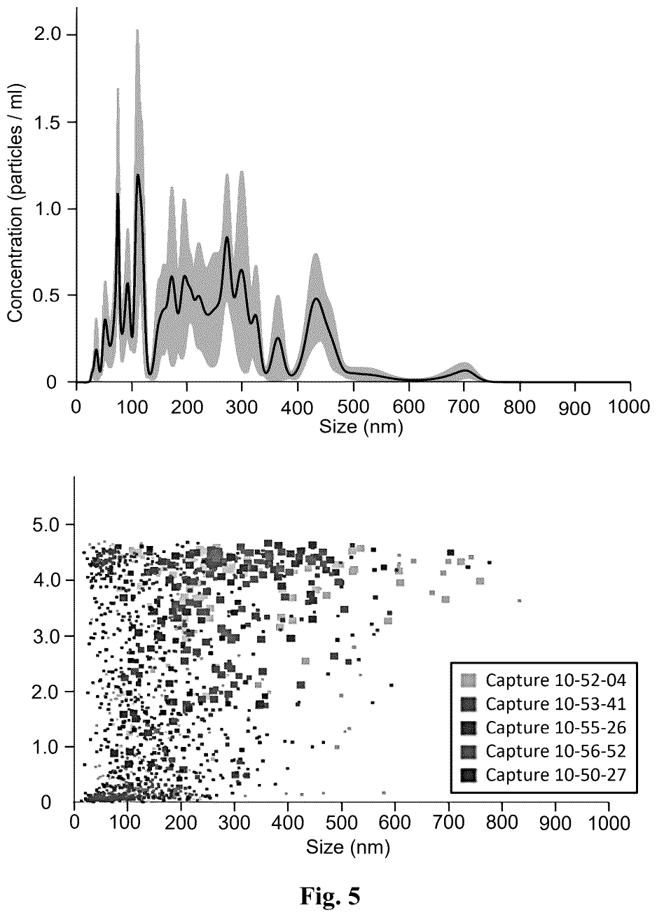

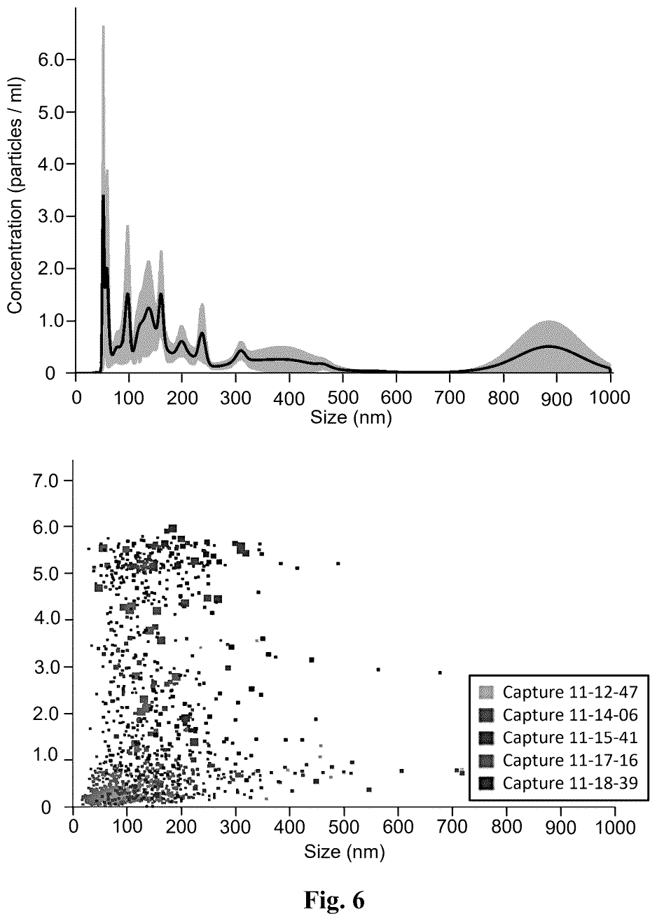

APC with genetic material encapsulated and attached to the outside of the capsule were also analyzed using NTA (FIG. 4 and FIG. 5). The NTA analysis of APC with encapsulated poly IC show that the size ranges from primarily 200-400 nm, with few particles larger or smaller (FIG. 4). The mean size for the APC with encapsulated poly IC was 270.7 nm with a standard error of 105.9 nm. The NTA analysis of APC with poly IC attached to the outside of the particle show that the size ranges from 100-300 nm, with few particles larger or smaller (FIG. 5). The mean size for the APC with poly IC attached to the outside was 246.3 nm with a standard error of 23.1 nm.

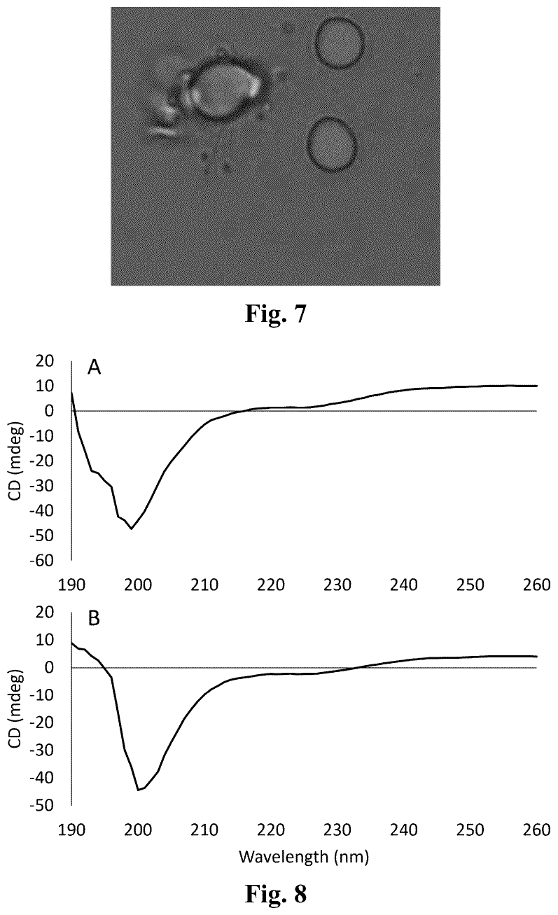

Rhodamine-6G dye was successfully encapsulated using APC prepared in pH 2.0 dilute HCl as confirmed by fluorescence spectroscopy and confocal microscopy. In the confocal microscopy image, a few APC containing rhodamine-6G dye can be seen (FIG. 7).

The dearth of capsules is due to the detection limit of the confocal microscope (1 micron) therefore, smaller assemblies cannot be observed. Light diffraction studies below, will show most of the capsules are far smaller. This result shows that the peptides form water filled capsules capable of trapping solutes.

### Stability Studies

APC capsules were analyzed for stability over time using DLS and Zeta potential measurements to track the changes in size and surface charge. Samples were stored at either room temperature (22° C.) or 4° C., in a refrigerator. Different samples including APC with encapsulated GFP plasmid and encapsulated GFP mRNA were analyzed over the course of 12-24 days. Each day, the samples were analyzed to determine their sizes, polydispersity, and surface charges.

The APC capsule with GFP plasmid encapsulated was stored at room temperature and analyzed over the course of two weeks. The initial size of the capsule was 389 nm (Table 5). Characteristics were tracked over the span of two weeks and stored at room temperature. Each sample was read three times and the results are the average of those runs, with the standard deviation included in the table for the size and hydrodynamic diameter.

The final reading of the capsule's size on the 13th day was 411 nm, showing the capsules initial and final readings of size remain relatively the same. However, the initial and final readings of the surface charge do differ substantially, with the first reading being 6.6 mV and the final reading being 0.5 mV (Table 6). Characteristics were tracked over the span of a month and stored at room temperature. Each sample was read two times and the results are the average of those runs, with the standard deviation included in the table for the size and hydrodynamic diameter.

TABLE 5
Characteristics of APC, with GFP plasmid encapsulated at a ratio of 1:10, formed
at pH 2 and brought up to pH 7 using imidazole buffer after 5 minutes.

| Day | DLS (nm) | Hydrodynamic Diameter (nm) | Polydispersity | Zeta (mV) |
|---|---|---|---|---|
| 1 | 245.92 ± 159 | 346.7 ± 143 | 24.40% | 17.37 |
| 2 | 284.83 ± 328 | 625.3 ± 510 | 27.90% | 12.23 |
| 6 | 460.4 ± 372 | 774.5 ± 712 | 25.00% | 5.23 |
| 7 | 239.38 ± 390 | 794.4 ± 898 | 25.37% | 10.23 |
| 8 | 260.48 ± 176.4 | 1686.4 ± 2030 | 34.17% | 0.53 |
| 11 | 1.105 ± 0.98 | 607.9 ± 296 | 27.85% | 0.25 |
| 15 | 27.32 ± 37.8 | 893 ± 861 | 32.70% | 0.65 |

TABLE 6
Characteristic of APC, with GFP mRNA encapsulated at a ratio of 1:10, formed at
pH 2 and brought up to pH 7 using imidazole buffer after 5 minutes.

| Day | DLS (nm) | Hydrodynamic Diameter (nm) | Polydispersity | Zeta (mV) |
|---|---|---|---|---|
| 1 | 241.5 ± 188 | 354.9 ± 66 | 25.55% | 22.6 |
| 2 | 121.025 ± 116 | 593 ± 82 | 23.80% | 21.4 |
| 5 | 21.835 ± 1.92 | 406.2 ± 8.9 | 27.60% | 6.3 |
| 6 | 25.65 ± 10.8 | 447.35 ± 29 | 29.50% | 14.8 |
| 11 | 30.205 ± 37 | 664.45 ± 25.6 | 26.45% | 6.4 |
| 12 | 73.945 ± 99.2 | 492.4 ± 230 | 30.40% | 0.7 |
| 22 | 27.82 ± 18.2 | 646.45 ± 230 | 28.80% | 4.5 |
| 24 | 150.28 | 1259.8 | 23.90% | 1.9 |

The stability of APC capsules containing encapsulated GFP mRNA was studied over the course of 24 days at both room temperature (20° C.) and at 4° C. (Tables 7 and 8). Characteristics were tracked over the span of a month and stored at 4° C. For Table 7, each sample was read two times and the results are the average of those runs, with the standard deviation included in the table for the size and hydrodynamic diameter. For Table 8, each sample was read three times and the results are the average of those runs, with the standard error of the mean included in the table for the size and hydrodynamic diameter.

TABLE 7
Characteristic of APC, with GFP mRNA encapsulated at a ratio of 1:10, formed
at pH 2 and brought up to pH 7 using imidazole buffer after 5 minutes.

| Day | DLS (nm) | Hydrodynamic Diameter (nm) | Polydispersity | Zeta (mV) |
|---|---|---|---|---|
| 1 | 46.39 ± 46.1 | 527.55 ± 193 | 29.90% | 20.1 |
| 2 | 109.01 ± 133 | 491 ± 62.3 | 25.70% | 21.1 |
| 6 | 216.045 ± 273 | 504.1 ± 130 | 20.20% | 10.3 |
| 7 | 90.075 ± 102 | 486.3 ± 84 | 28.30% | 10.8 |
| 8 | 97.705 ± 101 | 906.7 ± 106 | 23.55% | 5.6 |
| 11 | 87.33 ± 122 | 483.95 ± 2.89 | 27.75% | 0.8 |
| 15 | 14.97 | 262.9 | 31.10% | 0.5 |

TABLE 8
Properties of APC over the course of 16 days, stored at 4° C.

| | | Hydrodynamic | | Zeta |
|---|---|---|---|---|
| Day | Size (nm) | Diameter (nm) | Polydispersity | Potential (mV) |
| 1 | 259.44 ± 81.2 | 352 ± 20.7 | 28.70% | 3.6 |
| 2 | 278.85 ± 102 | 570.6 ± 79.4 | 33.96% | 4.7 |
| 3 | 359.78 ± 150.78 | 693.5 ± 142 | 23.06% | 0.533 |
| 4 | 123.31 ± 4.903 | 682.8 ± 98.6 | 28.33% | 0.167 |
| 5 | 133.58 ± 19.5 | 2272.7 ± 576 | 37.80% | 0.033 |
| 8 | 237.46 ± 60.5 | 3872.5 ± 3470 | 36.26% | 0.833 |
| 9 | 168.22 ± 83 | 707.6 ± 178 | 18.56% | 4.467 |
| 10 | 193.06 ± 70.1 | 932.3 ± 459 | 34.96% | 8.867 |
| 11 | 318.03 ± 181 | 728.3 ± 258 | 85.23% | 3.133 |
| 13 | 84.73 ± 4.73 | 651.5 ± 152 | 37.40% | 6.367 |
| 14 | 26.66 ± 25.93 | 23738.8 ± 12351 | 42.40% | 2.667 |
| 16 | 480.03 ± 143 | 13377 ± 5335 | 38.63% | 0.8 |

The size of the APC capsules began at approximately 108 nm for the sample stored at room temperature. On the 24th day of sitting at room temperature, the size of the APC capsules was 150 nm. Overall, the size of the APC capsules did not fluctuate substantially over time, however, the surface charge did change more drastically over time. The first reading showed a surface charge of 19.7 mV, while the final reading showed a surface charge of 1.9 mV. The surface charge of the sample at 4° C. fluctuated as well, beginning at 15.9 mV, and having a final reading at −0.3 mV. These results show that the APC may be stable at both 4° C. and room temperature (20° C.).

In addition to the decreases in surface charge over time the polydispersity and the hydrodynamic diameters of the assemblies increased, suggesting aggregation. Since cellular uptake is dependent on both surface charge and diameter these assemblies would not be efficient in delivering their cargos would not be delivered intracellularly.

The stability studies for the Ac-KKKFLIVIKKK-CONH2 (SEQ ID NO:6) sequence reveals that they are stable for only a few days when prepared and stored at either room temperature or 4° C. This instability limits their application to samples prepared just before using. In an attempt to improve APC stability, the longer Ac-KKKFLIVIGSIIKKK-CONH2 (SEQ ID NO:7) sequence was tested. It was reasoned that the longer hydrophobic sequence would increase stability. In Table 9 this longer sequence formed stable APC when formed at pH 3.5. Assembly at pH 2.0 yielded mixed results and required longer inoculation times that would have destroyed any RNAs prior to full encapsulation. Assembly occurs quickly at this pH within a minute.

TABLE 9
Results of the Ac-KKKFLIVIGSIIKKK-CO-NH₂ (SEQ ID NO: 7) APC
formed at a pH of 3.5. Three different times the APC were incubated at
pH 3.5 before the pH was increased to a neutral pH of 7, were analyzed.

| Assembly Time | | Hydrodynamic | |
|---|---|---|---|
| (minutes) | Size (nm) | Diameter (nm) | Polydispersity |
| 1 | 148.0 ± 97 | 687.0 ± 122.0 | 29.7% |
| 5 | 264.0 ± 100.4 | 490.2 ± 64.08 | 37.5% |
| 20 | 38.04 ± 11.614 | 4702 ± 4231 | 30.1% |

Having an assembly of pH 3.5 prevents autodegradation of mRNAs21 during the encapsulation step. The 1 min incubation time produced the smallest capsules. All subsequent experiments used the 1 min incubation time prior to PH neutralization.

The stability of the capsules prepared with the larger Ac-KKKFLIVIGSIIKKK-CONH2 (SEQ ID NO:7) sequence were studied at both RT and 4° C. Table 10 shows the stability at RT. Based on the polydispersity values observed these capsules are stable for about 7 days. The sizes did not show appreciable increase, however, from day eight and on the hydrodynamic diameters began increasing.

TABLE 10
Properties of Ac-KKKFLIVIGSIIKKK-CO-NH₂
(SEQ ID NO: 7) APC formed in 1 min over the course
of 13 days, stored at room temperature (22° C.).

| | Hydrodynamic | | |
|---|---|---|---|
| Day | Diameter (nm) | Size (nm) | Polydispersity |
| 1 | 707.0 ± 205 | 25.2 ± 2.52 | 23.3% |
| 2 | 532.6 ± 41.1 | 147.5 ± 65.3 | 49.1% |
| 3 | 540.0 ± 33.2 | 147.9 ± 65.7 | 72.5% |
| 5 | 605.1 ± 123.3 | 249.2 ± 33.72 | 145% |
| 6 | 447.0 ± 13.4 | 336.5 ± 30.66 | 14.5% |
| 7 | 477.4 ± 27.5 | 363.7 ± 65.2 | 26.8% |
| 8 | 2097 ± 744 | 177.5 ± 21.1 | 149% |
| 9 | 15000 ± 5409 | 92.41 ± 73.2 | 59.0% |
| 13 | 595.2 ± 56.8 | 161.2 ± 59.8 | 36.6% |

Each sample was read three times using dynamic light scattering to determine size, hydrodynamic diameter, and polydispersity. The results are the average of those runs, with the standard error of the mean included in the table for the size and hydrodynamic diameter.

The stability of the larger peptide capsules was also tested at 4° C. (Table 11). Lowering the temperature affords an extra week of stability for these preparations. Sie s average around 200 nm with polydispersion indices remaining the mid 20 percent range. The hydrodynamic range for those readings that did not have high standard of the mean were in the 400-500 nm range. Keeping these assemblies at the lower temperature definitely improved their viability. Each sample was read three times using dynamic light scattering to determine size, hydrodynamic diameter, and polydispersity. The results are the average of those runs, with the standard error of the mean included in the table for the size and hydrodynamic diameter.

TABLE 11
Properties of Ac-KKKFLIVIGSIIKKK-CO-NH₂ (SEQ ID NO: 7)
APC formed in 1 min over the course of 18 days stored at 4° C.

| | Hydrodynamic | | |
|---|---|---|---|
| Day | Diameter (nm) | Size (nm) | Polydispersity |
| 1 | 433.3 ± 57.1 | 220.3 ± 35.5 | 27.4% |
| 2 | 390.4 ± 35.5 | 22.3 ± 5.83 | 22.2% |
| 3 | 3916 ± 3522 | 21.8 ± 4.44 | 21.2% |
| 5 | 1360 ± 836 | 186.2 ± 37.5 | 31.0% |
| 6 | 464.6 ± 44.2 | 227.2 ± 48.5 | 14.5% |
| 7 | 598.7 ± 68.4 | 387.1 ± 66.8 | 20.0% |
| 8 | 507.6 ± 37.5 | 320.6 ± 43.7 | 23.4% |
| 9 | 544.9 ± 74.2 | 225.7 ± 101 | 23.2% |
| 13 | 1659 ± 1184 | 178.8 ± 77.0 | 36.3% |
| 14 | 770 ± 168 | 260.8 ± 137 | 13.8% |
| 15 | 904.7 ± 319 | 435 ± 161 | 16.9% |
| 16 | 311.5 ± 75.7 | 204.2 ± 24.8 | 63.5% |
| 18 | 727.3 ± 114 | 321.2 ± 45.6 | 43.4% |

To investigate factors that might be contributing to the observed increased stability circular dichroism studies were performed (FIG. 8) The spectra recorded at both the assemble and then neutralization pH values reveal nearly identical random coil secondary structures. This result indicates that the increased stability of this preparation is due to the presence of additional hydrophobic residues in the sequence thereby promoting additional Van der Waal interactions.

### GFP Plasmid Transfection

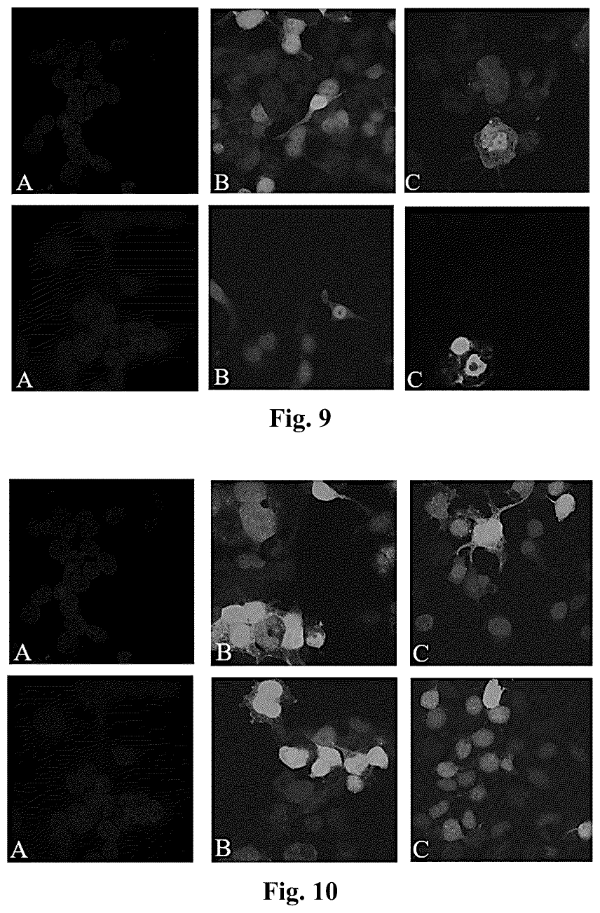

The confocal images in FIG. 9 show the results of the GFP plasmid transfection with APC, for the 1:20 outside (B) and 1:20 inside (C) conditions, as well as for the cell only (A). These confocal images were taken on a confocal LSM 700 laser-scanning microscope and analyzed using Zen blue photo editing to analyze the images. The HEK cell only condition, stained with DAPI can be seen expressing no GFP autofluorescence (A). The HEK cells treated with the 1:20 outside APC condition (B) and 1:20 inside APC condition (C) can both be seen expressing GFP (FIG. 9). Both the 1:20 outside (B) and 1:20 inside (C) conditions show GFP expression in the HEK cells (FIG. 9). While both conditions show GFP expression, the 1:20 outside (B) condition had a much higher confluence of HEK cells than did the 1:20 inside condition (C). Still, the GFP expression in both conditions indicates that the APC capsules were able to enter the HEK cells and deliver the GFP plasmid, allowing the for expression of GFP to occur.

The confocal images in FIG. 10 show the results of the GFP plasmid transfection for the 1:10 outside APC (B) and 1:10 inside APC conditions (C), as well as for the cell only (A). The condition with HEK cells only can be seen expressing blue from DAPI stain, but no GFP fluorescence. The 1:10 outside APC (B) condition, that is stained with DAPI and can be seen expressing GFP (FIG. 10). The 1:10 inside APC condition (C), that is stained with DAPI and can be seen expressing GFP (FIG. 10). Compared to the 1:20 conditions seen in FIG. 9, the 1:10 conditions in FIG. 10 show much higher expression of GFP fluorescence in the HEK cells. The confluence of HEK cells in both the 1:10 outside and 1:10 inside conditions are both high and relatively the same in both.

The expression of GFP in both the 1:10 outside and inside conditions indicates that at these conditions, the HEK cells were able to express the GFP plasmid delivered to them through the APC capsules. Both when the GFP plasmid was encapsulated and when the GFP plasmid was attached to the outside of the capsule showed the ability to deliver the genetic material to the HEK cells for expression.

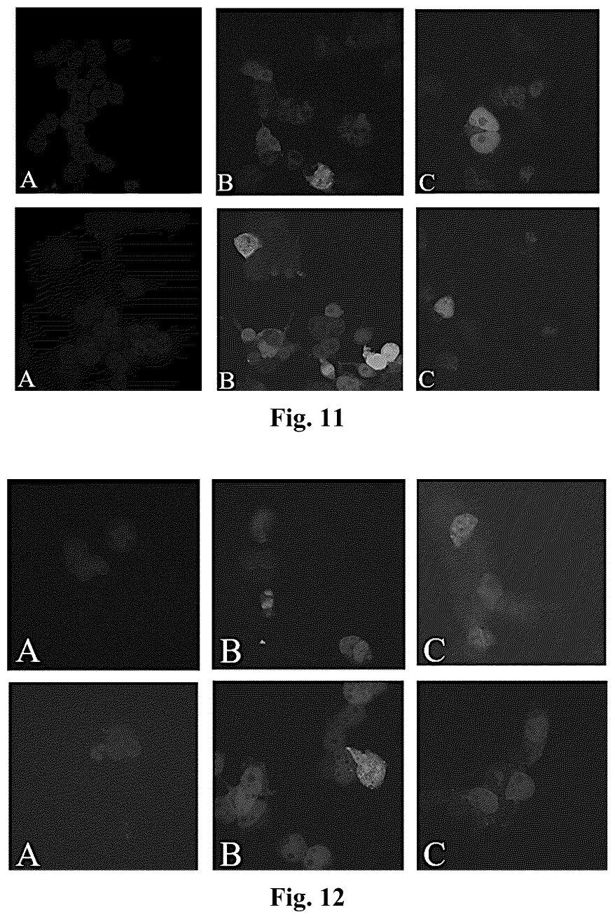

The confocal images in FIG. 11 show the results of the GFP plasmid transfection for the 1:5 outside (B) and 1:5 inside (C) APC conditions, as well as for the cell only (A). The left column shows the results for the HEK cell only condition, stained with DAPI. The 1:5 outside APC condition (B), that is stained with DAPI and can be seen expressing some GFP fluorescence. The 1:5 inside condition (A), that is stained with DAPI and can be seen expressing GFP fluorescence.

GFP mRNA Transfection

The confocal images in FIG. 12 show the results of the GFP mRNA transfection for the 1:20 outside (B) and 1:20 inside APC (C) conditions, as well as for the cell only (A). These images, like those in the plasmid transfection results, were taken on a confocal LSM 700 laser-scanning microscope and analyzed using Zen blue edition photo editing software. The HEK cell only condition (A), stained with DAPI expresses no GFP fluorescence (FIG. 12). The 1:20 outside APC condition (B) is stained with DAPI and can be seen expressing some GFP (FIG. 12). The 1:20 outside condition (C) is stained with DAPI and can be seen expressing GFP (FIG. 12).

The confocal images in FIG. 13 show the results of the GFP mRNA transfection for the 1:10 outside (B) and 1:10 inside APC (C) conditions, as well as for the HEK cell only (A). The HEK cell only condition (A) is stained with DAPI and is not expressing any GFP fluorescence (FIG. 13). The 1:10 outside condition (B) is stained with DAPI and can be seen expressing some GFP in a few cells (FIG. 13). The 1:10 inside APC condition (C) is stained with DAPI and can be seen expressing some GFP (FIG. 13). The cells in the 1:10 inside conditions were much more confluent than the other two conditions, therefore, a smaller percentage of cells in the confocal image are expressing GFP fluorescence.

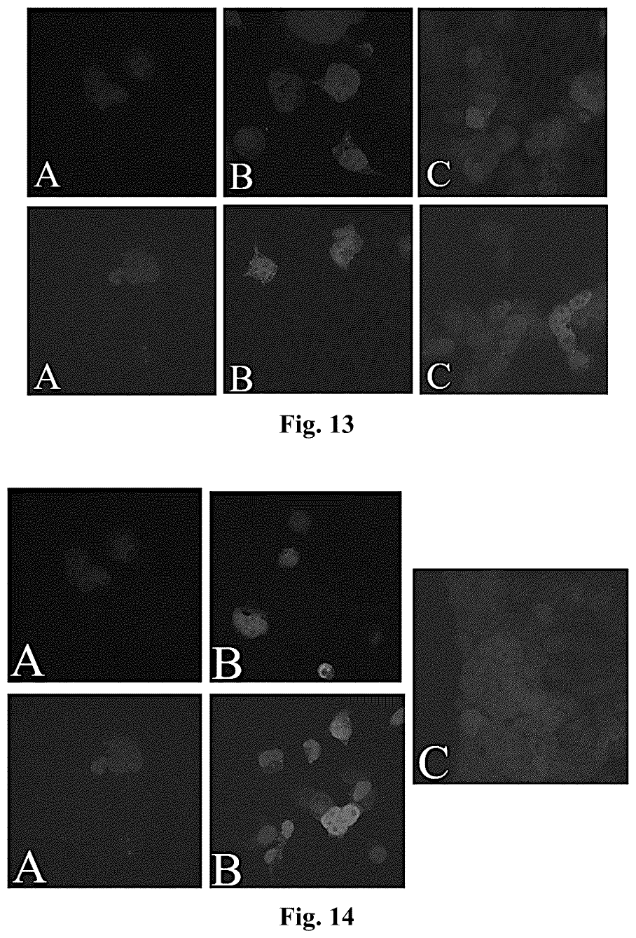

The confocal images in FIG. 14 show the results of the GFP mRNA transfection for the 1:10 outside (B) and 1:10 inside APC (C) conditions, as well as for the cell only (A). The HEK cell only condition (A) stained with DAPI, expressing no GFP fluorescence (FIG. 14). The 1:10 outside condition (B) is stained with DAPI and can be seen expressing some GFP in a few cells (FIG. 14). The 1:10 inside condition (C) is stained with DAPI and can be seen expressing some GFP (FIG. 14). The cells in the 1:10 inside conditions (C) were much more confluent than the other two conditions, therefore, a smaller percentage of cells in the confocal image are expressing GFP fluorescence. These results show that both GFP mRNA encapsulated in the APC and attached to the outside were able to be taken up and expressed by the HEK cells (FIGS. 12, 13, and 14).

### RiboGreen Assay

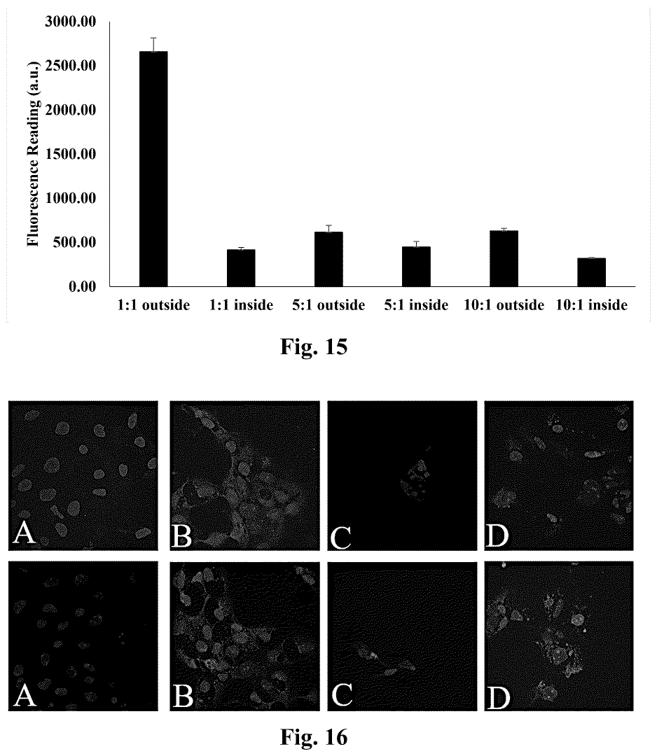

The results of the RiboGreen assay revealed that an equivalent ratio of 1:1, the difference between the encapsulated genetic material and genetic material attached to the outside of the APC capsule is the greatest. At this ratio, a significant difference between the fluorescence reading of the 1:1 outside and 1:1 inside APC can be observed (FIG. 15). The RiboGreen reagent fluoresces intensely when exposed to genetic materials, therefore, the more intense fluorescence reading of the 1:1 outside APC capsule indicates that genetic material was detected in the sample. For the 1:1 inside APC capsule, the low fluorescence reading indicates that the genetic material may be encapsulated as little genetic material was detected by the RiboGreen reagent. This result for the 1:1 conditions suggest that the internal packaging of the dsRNA is efficient. Only a slight difference can be observed in the 5:1 and 10:1 ratios of genetic material to peptide. At these ratios, the outside conditions have just a slightly higher fluorescence reading than the inside conditions. There is the possibility that some dsRNA remains on the surface after self-assembly, explaining why some fluorescence is detected in each of the inside conditions.

APC GFP mRNA Transfection Efficacy Results

Based on the confocal microscopy images shown in FIG. 16 the optimal conditions for mRNA transfection using APC capsules are to prepare the APC at pH 2 and wait 1 minute before increasing the pH to 7 using imidazole buffer (B). These images show the highest amount of the GFP expression in the HEK cells (B). In FIG. 16, the leftmost images show only The HEK cells stained with DAPI, which had not been treated with APC show no GFP expression (A). These images show the highest amount of the GFP expression in the HEK cells. There is no GFP expression in HEK cells when the APC were formed at pH 2 and incubated for 5 minutes before increasing the pH to 7 (C). The HEK cells treated with APC containing GFP mRNA, that were formed at pH 2 and incubated for 20 minutes before increasing the pH 7 show GFP expression (D).

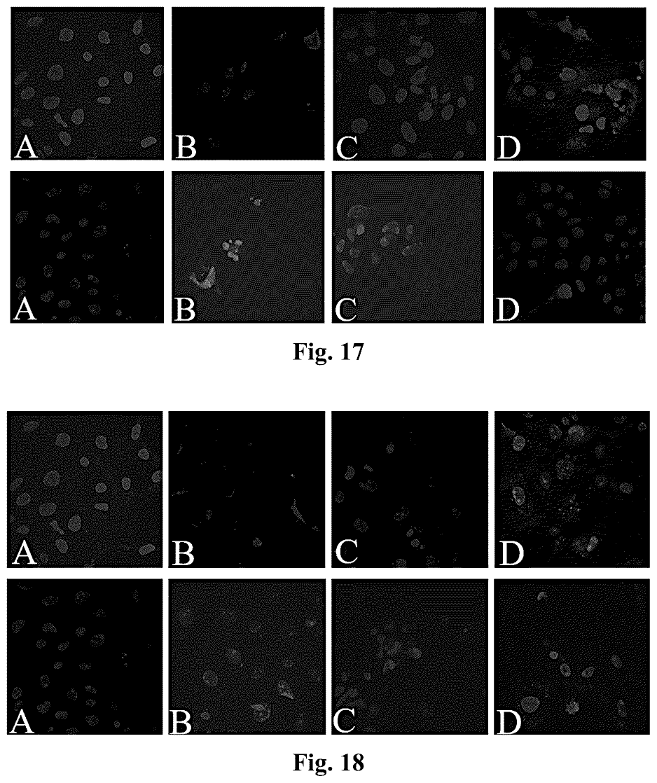

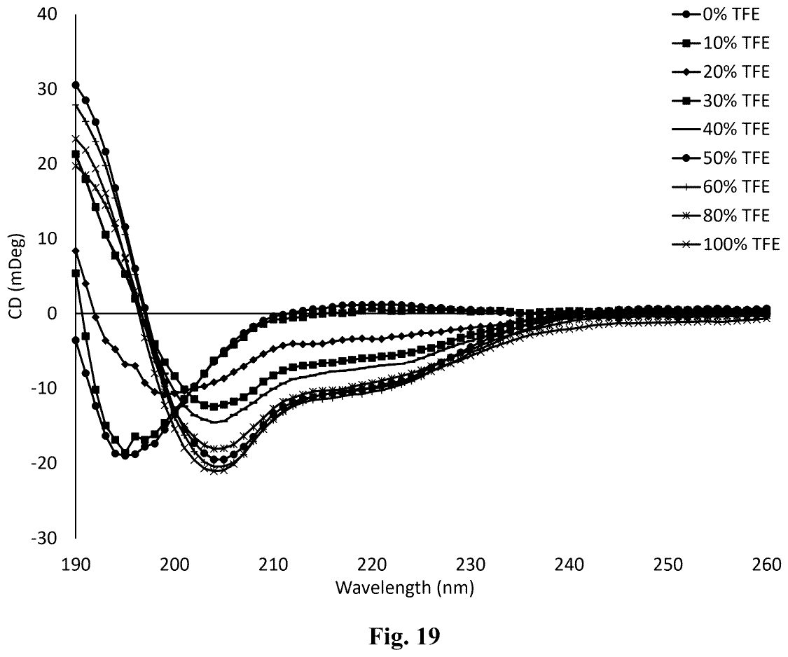

The most GFP expression and greatest confluence of HEK cells can be seen in the 1-minute incubation time. These same incubation times were repeated with formation of APC at pH 3 and pH 3.5, however, the results showed little GFP expression (FIG. 17 and FIG. 18). Out of these conditions, the pH 3.5 with 20-minute incubation condition showed the greatest amount of GFP expression. Overall, these results show the APC can deliver GFP mRNA to the HEK cells for expression.

### Conclusion

Circular dichoism was used to assess APC secondary structure at different pH values, revealing that when formed at pH 2 the APC capsules form a random coil. When initially formed at pH 2 and later raised to pH 7, the APC are shown to maintain a random coil secondary structure. This observation implies that once assembled at low pH, the secondary structure becomes locked and resistant to change when moved to elevated pH values. Studying the effects exposing of increasing concentrations of TFE to APC shows that the APC are held together by hydrophobic interations that can be disrupted by TFE concentrations>30% (FIG. 19). Below this concentration the secondary structure is a random coil, however, as the TFE concentration is increased to 30% the secondary structure switches to an α-helix. Most α-helices are monomeric, thereby implying that the APC prepared at pH 2 has dis-assembled at these higher TFE concentrations. The ability to open up the APC using TFE could allow for the quantfication of encapsulated solutes in future experiments. These self-assembled capsules have sizes of approximately 100-200 nm, as confirmed by both DLS and NTA analysis. The surface charge of the APC capsules is positive, due to hydrophilic lysine residues lining the outside of the capsules as illustrated in FIG. 1A.

The cationic surface allows for the electrostatic attachment of anionic molecules such as DNA and RNA to the outside of the APC capsules. The APC capsules are also capable of encapsulating anionic molecules. These capsules are readily taken up by cells where they are opened to release the encapsulated solutes. Surface bound materials are also released inside the cells and show uptake of encapsulating genetic materials. In vitro cellular of the APC capsules with genetic material was confirmed through transfection of HEK cells with GFP plasmid and GFP mRNA. These results suggest that two different types of molecules could be delivered, one inside and the other on the outside. An example of this might be the Crispr Cas9 enzyme inside and the RNA guides on the outside. The ability to deliver both to the same cell could enhance gene editing.

### Supplemental Data

TABLE 12
DLS and Zeta characterization of the APC from the GFP plasmid
transfection. Each sample was read three times and the
results are the average of those runs, with the standard deviation
included in the table for the size and hydrodynamic diameter.

| | | Hydrodynamic | | Zeta |
|---|---|---|---|---|
| Sample | Size (nm) | Diameter (nm) | Polydispersity | (mV) |
| 1:20 Outside | 307.56 ± 213.2 | 340.7 ± 115.4 | 24.00% | 4.4 |
| 1:20 Inside | 171.62 ± 110 | 474.2 ± 171.4 | 33.00% | 7.8 |
| 1:10 Outside | 159.7 ± 101.4 | 306.8 ± 83.3 | 28.20% | 1.7 |
| 1:10 Inside | 170.9 ± 167.7 | 1195 ± 720 | 28.00% | 2.0 |
| 1:5 Outside | 113.9 ± 77.4 | 546.8 ± 155.3 | 25.10% | 8.2 |
| 1:5 Inside | 199.4 ± 12.45 | 274.8 ± 86.3 | 24.87% | 4.4 |

TABLE 13
DLS and Zeta characterization of the APC from the GFP mRNA
transfection. Each sample was read three times and the results are
the average of those runs, with the standard deviation included
in the table for the size and hydrodynamic diameter.

| | Size | Hydrodynamic | | Zeta |
|---|---|---|---|---|
| Sample | (nm) | Diameter (nm) | Polydispersity | (mV) |
| 1:20 Outside | 217.5 | 422.6 | 27.00% | 2.9 |
| 1:20 Inside | 137.31 | 1462.4 | 31.00% | 7.3 |
| 1:10 Outside | 27.77 | 917.1 | 26.80% | 11.6 |
| 1:10 Inside | 810.2 | 2185 | 24.70% | 8.3 |
| 1:5 Outside | 36.81 | 1897 | 36.80% | 2.2 |
| 1:5 Inside | 41.13 | 2729 | 40.70% | 9.0 |

TABLE 14
DLS and Zeta characterization of the APC
from the GFP mRNA efficacy transfection.

| | Incubation | | | | |
|---|---|---|---|---|---|
| | Time | | Hydrodynamic | Poly- | Zeta |
| pH | (min) | Size (nm) | Diameter (nm) | dispersity | (mV) |
| 2 | 1 | 593.9 | 1287.8 | 9.20% | 0.1 |
| 2 | 5 | 63.78 | 1928.2 | 4.00% | 0.2 |
| 2 | 20 | 221.7 | 1311 | 32.60% | 0.1 |
| 3 | 1 | 0.36 | 1095.5 | 15.50% | 1.0 |
| 3 | 5 | 1183.1 | 3170 | 27.20% | 10 |
| 3 | 20 | 1476.4 | 1583 | 16.90% | 6.4 |
| 3.5 | 1 | 565.7 | 1011.9 | 9.40% | 1.6 |
| 3.5 | 5 | 240.3 | 1471.1 | 18.40% | 0.2 |
| 3.5 | 20 | 105.74 | 1068.2 | 38.10% | 2.1 |

After encapsulating the dye, APC were analyzed on DLS both before and after drying and rehydration. The size, polydispersity, and Zeta potentials remained similar both before and after drying and rehydration, with both being near 20 mV (Table 15). The size remained around 500 nm and the Zeta potential remained close to 20 mV (Table 15).

TABLE 15
DLS and Zeta potential analysis of APC with encapsulated rhodamine-
6G dye before and after drying and rehydrating the sample.

| | Size | Hydrodynamic | | Zeta Potential |
|---|---|---|---|---|
| | (nm) | Diameter (nm) | Polydispersity | (mV) |
| Before drying | 580.3 | 892.3 | 31.70% | 17.9 |
| After drying and | 622.4 | 3418 | 24.90% | 20.4 |
| rehydration | | | | |
| Before drying | 580.3 | 892.3 | 31.70% | 17.9 |
| After drying and | 622.4 | 3418 | 24.90% | 20.4 |
| rehydration | | | | |

TABLE 16
Ac-KKKFLIVIGSIIKKK-CO-NH₂ (SEQ ID NO: 7) incubated
at room temperature for either 1 minute, 5 minutes, or 20
minutes at pH 3.5, before increasing the pH to 7

| | | Hydrodynamic | |
|---|---|---|---|
| APC Formation | | Diameter | |
| Time (minutes) | Size (nm) | (nm) | Polydispersity |
| 1 | 148.0 ± 97 | 687.0 ± 122.0 | 29.7% |
| 5 | 264.0 ± 100.4 | 490.2 ± 64.08 | 37.5% |
| 20 | 38.04 ± 11.614 | 4702 ± 4231 | 30.1% |

TABLE 17
Ac-KKKFLIVIGSIIKKK-CO-NH₂ (SEQ ID NO: 7)
incubated at room temperature for either 1 minute, 5 minutes,
or 20 minutes at pH 2, before increasing the pH to 7

| | | Hydrodynamic | |
|---|---|---|---|
| APC Formation | Size | Diameter | Poly- |
| Time (minutes) | (nm) | (nm) | dispersity |
| 1 | 86.68 | 385.8 | 31.50% |
| 5 | 29.18 | 551.0 | 23.60% |
| 20 | 321.7 | 283.3 | 23.40% |

TABLE 18
Size and Charge Characteristics of Ac-KKKFLIVIGSIIKKK-CONH2
(SEQ ID NO: 7) APC Sequence. Characteristics of 15 residue
APC sequence formed at different pH values. Hydrodynamic
diameter and size shown with standard deviation and n = 3.

| | | | | Zeta |
|---|---|---|---|---|
| | | Hydrodynamic | | Potential |
| pH | DLS (nm) | Diameter (nm) | Polydispersity | (mV) |
| 2 | 143.2 ± 210 | 812.1 ± 655 | 33.00% | 5.7 |
| 3.9 | 282 ± 191 | 1515 ± 571 | 38.3% | 0.7 |
| 7 | 191.3 ± 160 | 681.1 ± 370 | 35.2% | −0.2 |
| 12 | 2479 ± 1419 | 1321.1 ± 102 | 33.8% | −6.8 |

As depicted in FIG. 1 with the shorter sequence, we see a similar assembly of nanofibrils with the longer peptide sequence when “assembled” at the neutral (~7) or elevated (~12) pH.

### Example 2

### APC Peptide Assembly Protocol

1. After synthesis and cleavage (and storage if applicable), dry peptides are dissolved in 2,2,2-trifluoroethanol>99% (TFE).
2. The concentration of the peptide solution is determined by measuring the absorbance of phenylalanine at a wavelength of 257.5 nm, on a CARY 50 Bio UV-visible spectrometer (Agilent Technologies, Santa Clara, CA).
3. The concentration of peptide in solution is then calculated using Beer's Law

$$A = ε cl$$

where A=absorbance, ε=molar extinction coefficient, c=concentration, and l=path length.
The path length was 0.3 cm, as absorbance measurements were taken using a 0.3 cm length quartz cuvette.
The molar extinction coefficient of phenylalanine at 257.5 nm was used for calculations, which is 195 M⁻¹ cm⁻¹.
Peptide final concentrations are typically prepared at 1.0-2.0 mg peptide per mL of TFE in an Eppendorf tube, unless another concentration or amount of peptide is specified.
4. Solvent is then evaporated using a Speed-Vac vacuum system for 20 min.
5. Once the peptide drying process is complete, the peptides are rehydrated with 20 μL of a low-pH buffer: 10 mM glycine-HCl buffer pH 2.0 (or 3.5).
6. The rehydrated peptides in low-pH buffer are incubated at room temperature for 1-20 minutes.
7. The pH of the solution is then raised to neutral pH of 7.0 by adding 20 μL of 10 mM Imidazole buffer at pH 7.0 with HCl. Imidazole buffer is prepared by dissolving solid imidazole in water and then adjusting the pH to 7.0 with the dropwise addition of concentrated HCl. That solution is added to the newly formed APC at low pH to bring them up to neutral pH.

### Example 3

### Introduction

Example 1 describes remarkable peptides having unique secondary structures found to be contingent upon the pH conditions of the environment. Notably, under acidic conditions with a pH of ~2, the peptides exhibited a remarkable property of self-assembling into nano-capsules having potential for applications in drug delivery, primarily owing to its capability to offer a cationic surface for binding nucleic acid or encapsulating therapeutic agents with great efficiency. Even when moved from low pH values to higher values of pH 7 or greater, the nano-spheric geometry remained intact, underscoring the suitability of the peptides for stable drug delivery applications.

This work describes a strategic modification of an original APC peptide sequence, resulting in the new sequence KKKFLIVIIVILKKK (SEQ ID NO: 56), designed with an extension and mirrored reflection of a segment of the original sequence. The central hydrophobic sequence FLIVI (SEQ ID NO:1) serves as the core, mirroring itself with the added residues IVIL (SEQ ID NO: 57). Within this extended structure, the peptide results in APCs having even further improved stability, while preserving the amphipathic nature of the original sequence.

### Materials and Methods

Solid-phase peptide synthesis—The modified peptide, Ac-KKKFLIVIIVILKKK-CONH₂ (SEQ ID NO:56) was synthesized using solid-phase peptide synthesis, as previously described in Example 1.

Preparation of APC—Peptides were dissolved either at low pH in Glycine-HCl-buffer as previously described in Example 1 or at neutral pH in Imidazole buffer (Tris Imidazole). Other neutral buffers derived from bases, such as nitrogen-based buffers, Tris-HCl or Borate buffered saline can be used. The modified peptides will form APCs under various pH ranges from pH 2 to 9. In either case, the peptides are stirred in the buffer system at room temperature/ambient conditions for about 10 to 20 minutes. The concentration of peptide was determined by measuring the absorbance of phenylalanine at a wavelength of 258 nm, on a CARY 50 Bio UV-visible spectrometer (Agilent Technologies, Santa Clara, CA). Final concentrations of 1 mM APC were prepared by adding peptide dissolved in TFE into an Eppendorf tube. Then, the solvent was removed using a vacuum and rotor. After drying, the APC peptides were rehydrated at different pH values ranging from 2 to 9 and incubated at room temperature for various times. Modified APCs prepared at low pH were subjected to a pH shift from low pH to pH to 7. The sizes of the peptide nanoparticles (capsules) were measured.

GFP mRNA expression by modified APC; surface bound and encapsulation—For experiments involving GFP mRNA transfection, both surface-bound mRNA and mRNA encapsulated were utilized in the modified APC. Various ratios of modified APC to GFP mRNA were tested, involving attachment to the surface and encapsulation within the modified APC. The tested ratios included 10:1, 20:1, and 50:1 weight-to-weight (w/w) ratio. All ratios maintained a consistent 200 ng of EGFP mRNA (TriLink Biotechnologies, San Diego, CA). The quantity of peptide varied, with 2 μg in the 10:1 ratio, 4 μg in the 20:1 ratio, and 10 μg in the 50:1 ratio. The precise volume of APC was calculated based on the concentration of the stock solution and the molecular masses of the peptides (Table 19).

TABLE 19
Original APC and modified APC peptide sequence

| Peptides | Sequence | MW (g/mol) |
|---|---|---|
| Original APC | KKKFLIVIKKK | 1413 |
| | (SEQ ID NO: 6) | |
| Modified APC | KKKFLIVIIVILKKK | 1853 |
| | (SEQ ID NO: 56) | |

HEK293 Cells were initially placed in 8-well chambers at a concentration of 80,000 cells per well, suspended in 0.5 mL of complete medium, and allowed to incubate. Following an overnight incubation, the culture medium was exchanged with Opti-MEM Reduced Serum (Thermo Fisher Scientific, Waltham, MA), incorporating modified APC-GFP mRNA complexes at various (w/w) ratios. The cells were then subjected to a 4 hr incubation at 37° C. with these complexes, after which they were replenished with fresh DMEM media. After a subsequent 48 hr period, cells underwent washing with phosphate-buffered saline (PBS), fixation using 200 μL of 4% paraformaldehyde (PFA) in PBS for 10 min at room temperature, and permeabilization with 0.1% Triton X for 1 hr (Thermo Fisher Scientific, Waltham, MA). Following this, cells were rinsed with PBS, and nuclei were stained using 4′, 6′-diamidino-2-phenylindole (DAPI) (Thermo Fisher Scientific, Waltham, MA). Evaluation of GFP transfection efficiency resulting from the treatment with modified APC-GFP mRNA complexes was performed utilizing an LSM 880 laser-scanning microscope (Carl Zeiss, Gottingen, Germany), with subsequent image analysis conducted through ZEN 3.5.

CD spectra measurement—CD spectra were obtained using a Jasco-815 spectrophotometer (Jasco Analytical Instruments, Easton, MD). Peptides modified with valine and isoleucine were prepared according to the Materials and Methods, For acidic conditions, 0.2 N HCl was utilized to adjust the pH, while basic samples were treated with IN NaOH buffer to achieve alkaline conditions. To evaluate mRNA encapsulation, peptides were meticulously dried using a vacuum evaporation system until reaching a final concentration of 0.1 mM upon rehydration with mRNA in H₂O at a specific pH. The concentration of mRNA in H₂O was adjusted based on the quantity of peptides to achieve a targeted encapsulation ratio of 50:1 (w/w). Similarly, for evaluating mRNA surface binding on modified APC, peptides underwent drying using a vacuum evaporation system to reach a final concentration of 0.1 mM upon rehydration with H₂O at a specific pH. Subsequently, mRNA was added to the rehydrated modified APC and adjusted according to peptide quantity to achieve the desired ratio of 50:1 (w/w). Nitrogen gas flowed through the CD spectrophotometer during runs. Spectra were collected using 1 mm quartz cuvettes (Starna Cells Inc., Atascadero, CA), scanning from 260 nm to 190 nm at 50 nm/min. Each sample underwent five scans, averaged, and smoothed.

Time stability studies—The stability of modified APC in aqueous solution was evaluated through Nanoparticle Tracking Analysis (NTA) measurements, following the procedures outlined in the “Nanoparticle Tracking Analysis (NTA)” section of the Materials and Methods, as detailed in preceding chapters. The stock sample of modified APC (1 mg/mL), stored at room temperature on a laboratory benchtop (25° C.), served as the subject of analysis. For each scheduled assessment date, 1 mL of the sample was extracted from the stock tube and subjected to the stability check. The temporal stability investigations were conducted at intervals of 1, 3, 6, 16, and 27 days subsequent to the production of the modified APC.

### Results

Surface-Bound and Encapsulated GFP mRNA Transfection Using Modified APC

The focus is on the examination and validation of modified APC's efficacy in mRNA delivery and transfection, employing GFP mRNA as the investigative tool. The study was methodically divided into two distinct applications to comprehensively assess the delivery dynamics. The surface-bound approach was explored first, where GFP mRNA is intricately tethered to the positively charged APC surface. Next, we examined an encapsulation strategy, encapsulating GFP mRNA within the aqueous core of the modified APC.

This dual-pronged approach not only provided a comprehensive analysis of the modified APC's mRNA delivery capabilities but also shed light on the potential applications and limitations of surface-bound and encapsulated strategies in optimizing transfection efficiency. The results obtained from these investigations contribute valuable knowledge to the broader field of gene and drug delivery vectors.

Before assessing GFP mRNA expression with modified APCs, it's essential to evaluate their capsule forming capacity when mRNA is either encapsulated within them or bound to their surface. NTA data reveals successful capsule formation in both scenarios, when nucleic acids are encapsulated (data not shown) and nucleic acids are bound on surface (data not shown), laying the groundwork for subsequent mRNA delivery assessments.

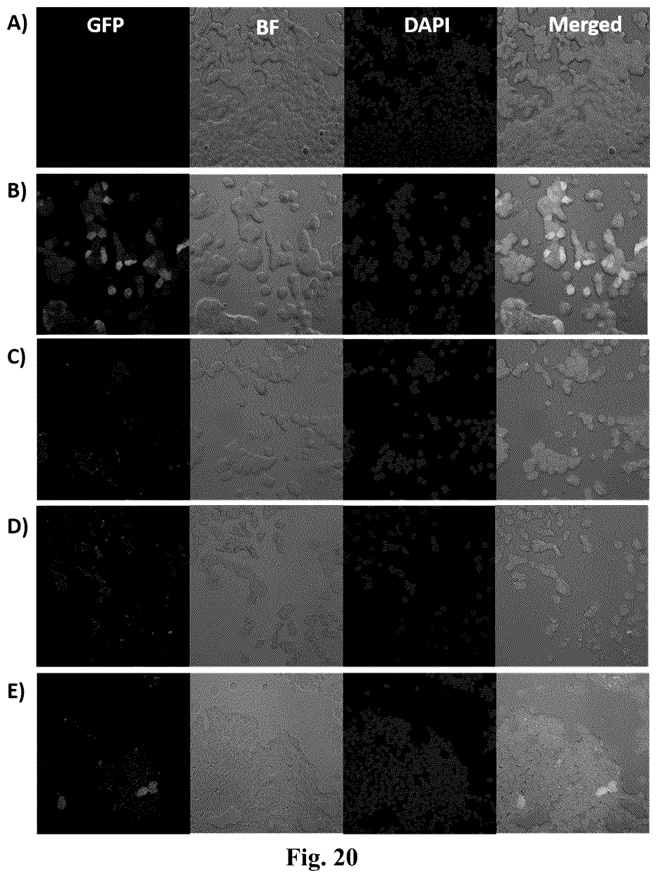

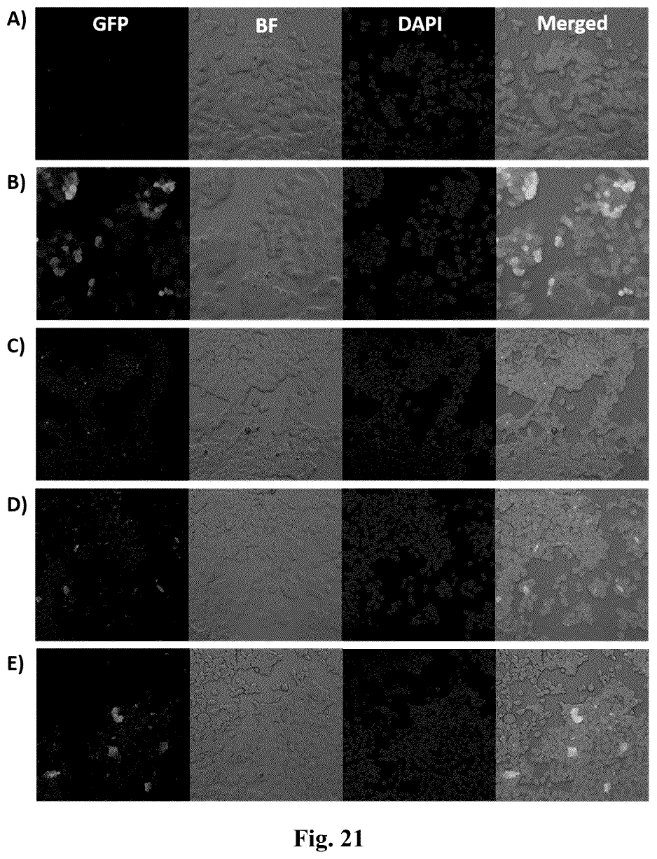

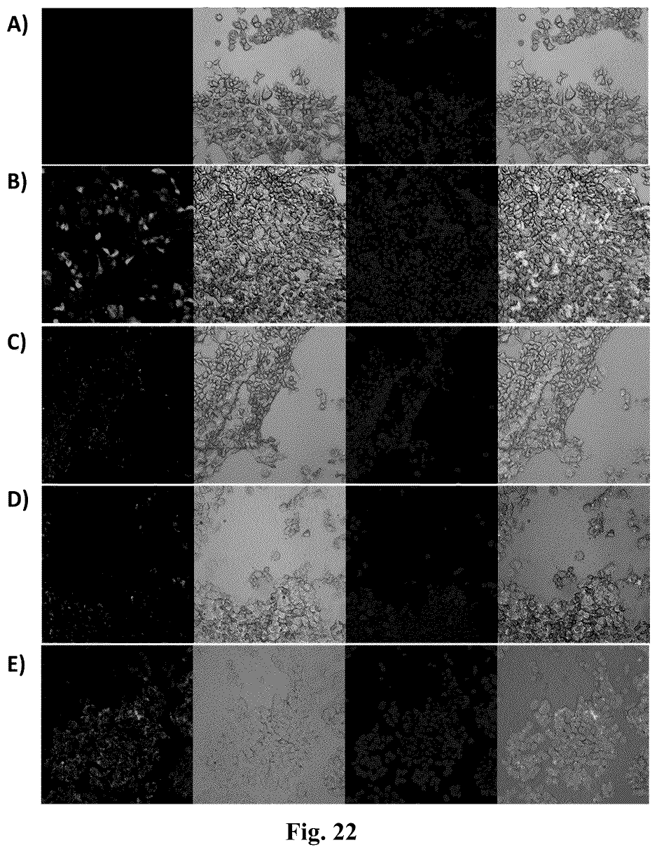

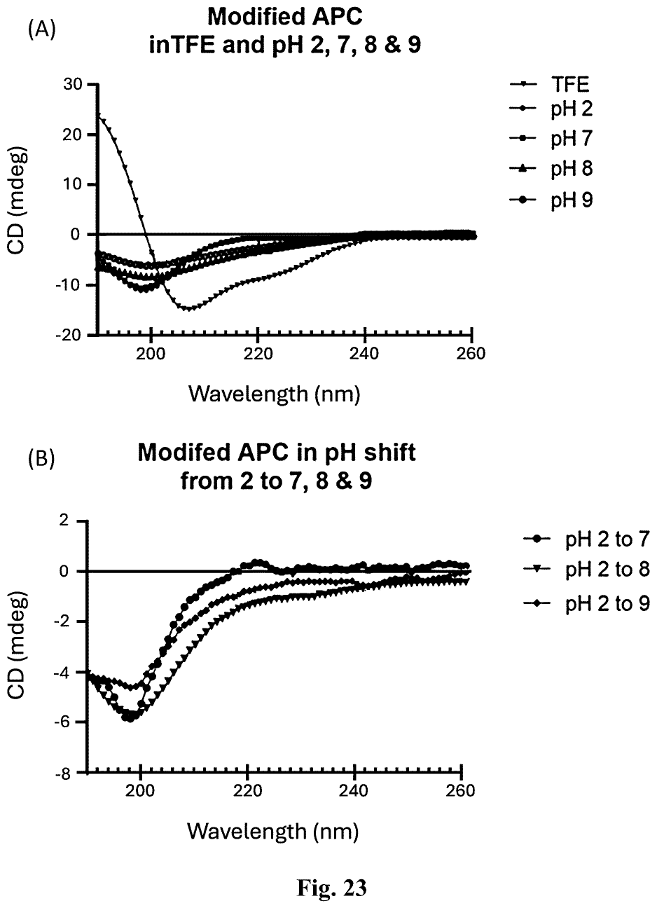

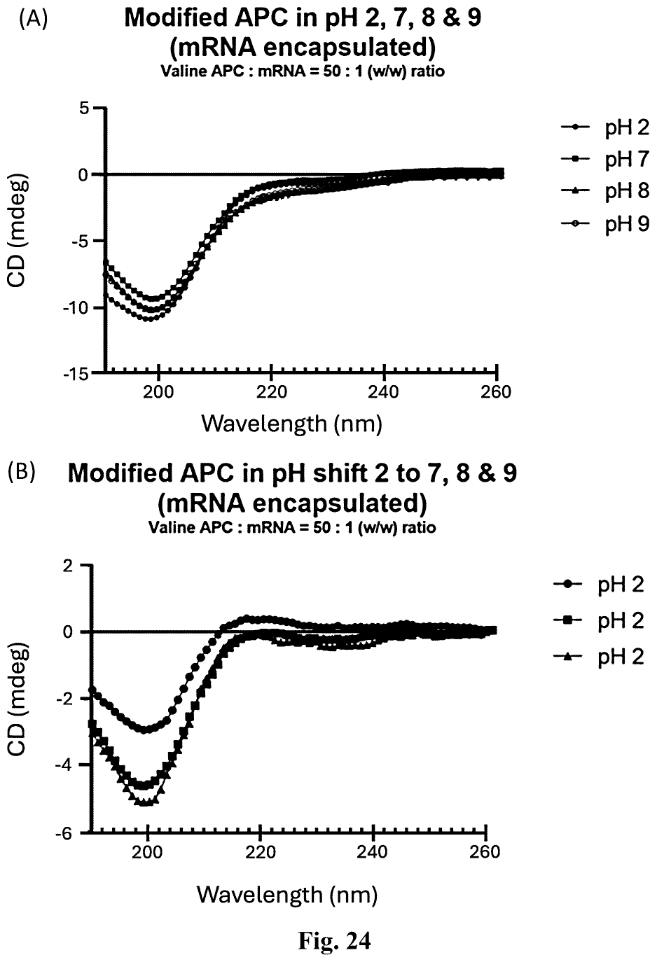

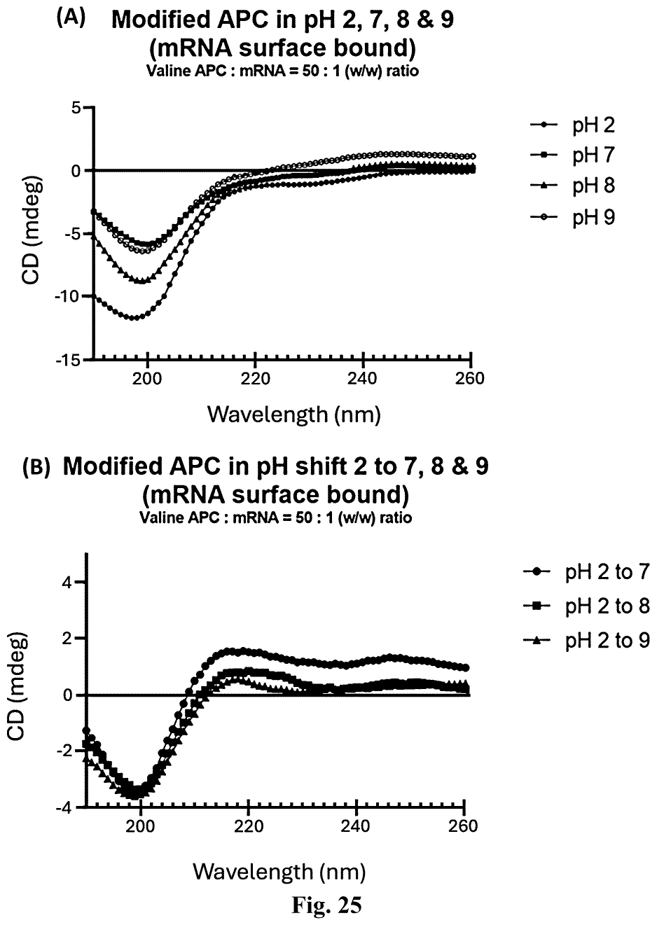

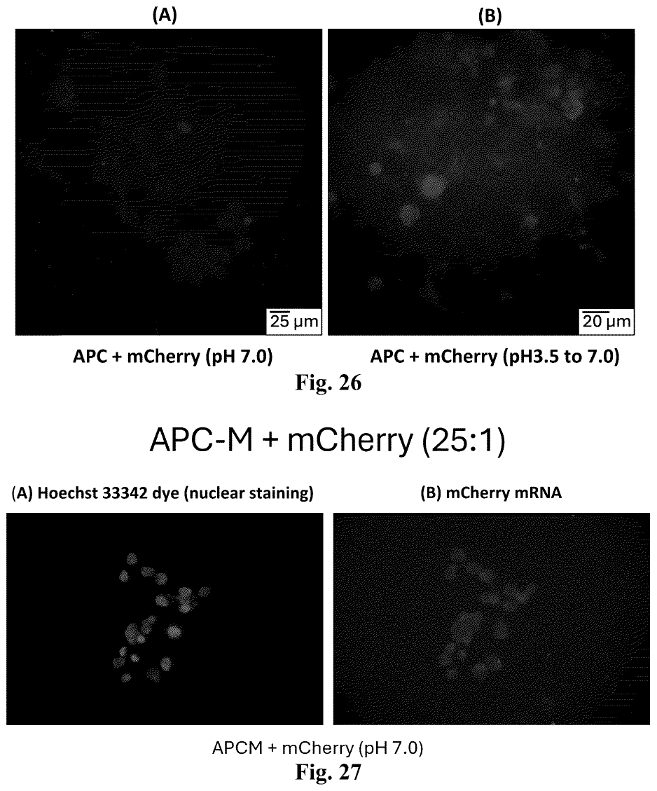

In FIG. 20, the results of surface-bound mRNA and gene transfection on HEK293 cells are displayed. A) served as the negative control, while B) shows the positive control (Lipofectamine 3000). C) demonstrated the effects at a modified APC to mRNA w/w ratio of 10:1, D) the outcome at a ratio of 20:1 and E) the experiment at a ratio of 50:1. At the 50:1 w/w ratio, a discernible increase in GFP expression was detected over the lower ratios.

Following surface-bound mRNA delivery by modified APC, we proceeded to employ encapsulated mRNA as cargo within the modified APC. As illustrated in FIG. 24, the modified APC exhibited a capsule shape, with the inner capsule providing space to accommodate mRNA for cellular delivery. The divisions in this analysis mirror those of the surface-bound version, presented in FIG. 20, ranging from a 10:1 to a 50:1 w/w ratio. FIG. 21 illustrates the GFP expression resulting from mRNA delivery encapsulated in modified APC. In FIG. 21A) and B), the negative control representing non-treated conditions and positive control (Lipofectamine 3000) are shown. C), D), and E) correspond to w/w ratios of 10:1, 20:1, and 50:1, respectively. This expression levels highlights the effectiveness of modified APC as a gene delivery vector, both in surface-bound and encapsulated configurations.

Various modified APC to mRNA w/w ratios were analyzed, observing higher GFP expression at the 50:1 ratio. Following surface-bound mRNA delivery, we explore encapsulated mRNA within modified APC, observing significantly higher GFP expression at the 50:1 ratio. This underscores the efficacy of modified APC in gene delivery, whether surface-bound or encapsulated.

### Stability Studies

NTA was used to assess the long-term stability of the modified APCs. In this study, the size of the nanoparticles was systematically tracked for about a month. To ensure the ideal temperature conditions for these measurements, NTA machine was carefully configured to operate at 25° C. and stock samples were stored at room temperature throughout the experiment, thereby maintaining the integrity of the experimental environment and ensuring the accuracy of the recorded data. NTA graphs are not shown. The NTA data for Days 1, 3, 6, 16 27 are provided in the Table below. Notably, the data revealed a consistent hydrodynamic size of the modified APC over the entire 27-day observation period (Table 20).

TABLE 20
Size stability of modified APC for 27 days at room temperature

| | Hydrodynamic | | | |
|---|---|---|---|---|
| | diameter (nm) | sd | Mode (nm) | sd |
| Day 1 | 175.8 | 1.6 | 141.8 | 8 |
| Day 3 | 171.8 | 5.1 | 136.2 | 7.9 |
| Day 6 | 143 | 2.1 | 116.2 | 7.8 |
| Day16 | 151.9 | 2.3 | 126.5 | 4.4 |
| Day 27 | 164.7 | 2.2 | 136.9 | 3.3 |

APC formation and monitoring display from Day 1 to Day 25 was also recorded (data not shown). The data demonstrated that for the diameter of the capsule, the peaks of the graphs are uneven, and the size distribution is not consistent throughout the experiment duration compared to the modified APC. This stability in size strongly suggests that these modified APCs exhibit exceptional thermodynamic stability. Furthermore, the NTA monitoring display indicates minimal particle aggregation throughout the monitoring period (data not shown), underscoring a high degree dispersion. These combined findings strongly support the conclusion that modified APCs demonstrate outstanding colloidal stability for a duration of approximately one month at a temperature of 25° C. This robust stability is a highly favorable attribute for their potential applications in various therapeutic and drug delivery contexts.

After assessing and comparing the capsule-forming capacity, aggregation, and stability, long term stored modified APCs were tested for their transfection capacity. FIG. 22 displays the GFP expression resulting from mRNA delivery with mRNA surface-bound on modified APCs. These modified APCs were stored at room temperature in aqueous solution for approximately two months before use. In FIG. 22A) and B), the negative control (representing non-treated conditions) and positive control (Lipofectamine 3000) are presented, respectively. FIG. 22 C), D), and E) correspond to w/w ratios of 10:1, 20:1, and 50:1, respectively. Similar to freshly prepared modified APCs, long-term stored modified APCs also show discernible GFP expression in HEK293 cells, especially at the 50:1 w/w ratio. These results highlight the transfection efficiency and stability of modified APCs as gene delivery vectors.

In addition to long-term stability, assessing pH stability is crucial in understanding the robustness of nano-carrier delivery systems. Variations in pH can profoundly influence the behavior and integrity of nanoparticles, particularly in physiological environments. Therefore, alongside the evaluation of long-term stability, we extended our analysis to include pH stability. This comprehensive assessment provides a deeper understanding of how modified APCs respond to variations in pH, further enhancing our ability to design delivery vectors that maintain efficacy across diverse biological conditions. CD was employed to assess the secondary structure of the modified peptides comprising APC across various pH values, as depicted in FIG. 23(A), including pH 2, 7, 8, and 9. At pH 2, the modified APC exhibited a random coil secondary structure, indicated by a distinct minimum in CD (mDeg) near 200 nm. Similarly, the modified APC formulated at pH 7, 8, and 9 also displayed a distinct minimum in CD (mDeg) at 200 nm, characteristic of a random coil secondary structure. Remarkably, even as the pH of the aqueous solution changed from 2 to 7, 8, and 9, transitioning from acidic to basic conditions, the random coil secondary structure was sustained (FIG. 23(B)). it is noteworthy that despite the shift in pH from acidic to basic conditions, the secondary structure of the modified APC remained unaffected, demonstrating robustness across varying pH environments.

Following the CD spectra measurement of modified APCs under different pH conditions, subsequent analyses were performed on mRNA encapsulated within the modified APCs and mRNA bound to their surface. These analyses specifically addressed the preservation of the modified APCs' secondary structure across varying pH environments while nucleic acids were either encapsulated or surface-bound. These investigations yielded pivotal insights into the stability of them during interactions with nucleic acids.

FIG. 24 illustrates mRNA encapsulated within modified APCs across various pH values. Remarkably, the modified APCs maintained a consistent random coil secondary structure irrespective of pH changes. This stability in secondary structure, akin to the observations without nucleic acid (FIG. 23), underscores the robustness of modified APCs.

Similarly, FIG. 25 provides additional evidence of the modified APCs' pH stability, showcasing their consistent random coil secondary structure even when mRNA is bound to their surface across different pH values. This robust maintenance of secondary structure suggests the modified APCs' resilience and suitability for effectively delivering genetic materials under diverse physiological conditions.

### Conclusion

The modified peptides preserve the amphipathic properties critical for self-assembly while introducing elements that contribute to enhanced stability as compared to first generation APCs. The resulting structure, characterized by a mirrored and extended reflection of the original sequence, holds promise for biomedical research, offering improved therapeutic strategies and innovative solutions. The comprehensive evaluation of the modified APC's gene delivery capacity, utilizing GFP mRNA and assessing both surface-bound and encapsulated approaches, showcases its versatility in optimizing transfection efficiency. The observed increase in GFP expression from 10:1 to 50:1 w/w ratios in surface-bound applications and consistent expression levels in encapsulated mRNA across various ratios affirm the stability and efficiency of the modified APC as a gene delivery vector. Furthermore, the meticulous assessment of the aqueous stability of modified APC through NTA reveals exceptional thermodynamic and kinetic stability over a 27-day period at 25° C. The absence of degradation or noticeable changes in size underscores the potential applications of these nano-carriers in various therapeutic and drug delivery contexts. In addition to long-term stability, the pH stability of modified APCs was evaluated through CD analysis, demonstrating consistent random coil secondary structure across various pH conditions. This robust stability under different pH environments further highlights their suitability as carriers for genetic materials in diverse physiological and relatively extreme pH conditions.

### Example 4

APC Transfection Protocol with Modified APCs (SEQ ID NO:56)

### Materials

1. Eukaryotic cells (HEK293T) to be transfected.
2. Freeze dried APC/mCherry PNP formulations (stored at −20° C. or colder).
3. One formulation was prepared in Imidazole buffer (IMB) pH 3.5
4. Second formulation was prepared in Imidazole buffer pH3.5 then pH shifted to neutral pH with 4-Volumes of IMB pH 8.0

### Protocol

1. The day before transfection, resuspend the cells gently by pipetting and count and seed the cells to reach 65 to 75% confluency by the time of transfection on the next day, dilute as needed to a cell density of ~0.5×10⁵-1×10⁵ cells/mL in the complete growth medium, and incubate the cells overnight
2. Transfection:
   a. Reconstitute the freeze dried mCherry/APCs transfection reagent (25:1) in 100 μL complete DMEM media.
   b. Remove the growth medium from the cells, and gently wash the cells once with DEME and the aspirate the wash media and discard.
   c. Add the prepared 100 μL mCherry/APC PNP transfection solution to the cells and gently rock the transfection solution over the cells.
   d. Incubate at 37° C. in a humidified, 5% CO₂ incubator for 2-4 hours.
   e. Dispense additional 400 μL of complete growth medium to each well, making the volume to 500 μL.
   f. Place cells back into the incubator for 24-26 hours.
3. Fix and Stain Cells for Imaging
   a. Aspirate the media and wash cells gently with 200 μL PBS pH 7.4 3X.
   b. Add 100 μL of 1:200 diluted Hoechst 33342 nucleic acid stain at room temperature for 15 minutes.
   c. Wash cells 3× with 200 μL PBS.
   d. Add 100 μL freshly prepared 2% cells fixing solution for 15 minutes at RT, and then remove and wash cells with 200 μL PBS, and allow to air dry briefly.
   e. Add mountant solution avoiding air bubbles, and cover slide with coverslip and allow to cure at room temperature in dark conditions for 18-24 hr, and then store at 4 C until imaging.
4. Evaluate Transfection Efficiency
   a. Inspect or image the cells with a fluorescence microscope.

### Results

Transfection was carried out using both first generation APCs and second generation APCs. As explained for the first generation APCs, for loading of mRNA, the mRNA interaction has to occur at low pH (2-3) followed by addition of a buffer to shift the final pH to ~7 to make the stable capsule around the mRNA that is ready for cell transfection. As can be seen in FIG. 26A and B, when prepared at pH 7, very little mCherry is encapsulated by the APCs. However, when prepared at pH 3.5 and then shifted to pH 7, a significant increase in encapsulated mCherry is observed.

Advantageously, with the modified (second generation) APCs, capsules can be loaded with mRNA at neutral pH (pH~7). As can be seen in FIG. 27, significant encapsulation of mCherry was obtained when the modified APCs were prepared at pH 7, and then used to transfect cells. This expands the types of agents that can be encapsulated and the conditions in which APCs can be prepared.

1. A peptide capsule comprising a membrane having an exterior surface and defining a liquid-receiving interior space configured to encapsulate water-soluble active agents therein, wherein the membrane consists of a plurality of linear peptides, each peptide comprising a hydrophobic core of between 4 and 12 hydrophobic amino acids flanked by N- and C-terminal hydrophilic segments each comprising between 3 to 4 hydrophilic amino acids.

2. The peptide capsule of claim 1, said capsule having a particle size from about 50 to about 500 nm, said capsule exterior surface having a positive surface charge.

3. The peptide capsules of claim 1, wherein said active agents are selected from the group consisting of nucleic acids, fluorescent dyes, peptides, therapeutic proteins, enzymes, hydrophilic small molecule compounds and drugs, sugars, poisons, and potentiators.

4. The peptide capsules of claim 1, further comprising a compound covalently or electrostatically attached to said exterior surface selected from the group consisting of as antibodies, antigens, receptor ligands, vitamins, and targeting moieties.

5. A method of delivering water-soluble active agents to an organism, said method comprising administering or applying a composition to said organism, said composition comprising a plurality of peptide capsules according to claim 1, in which said water-soluble active agents are encapsulated.

6. The method of claim 5, wherein said organism is a plant, wherein said administering or applying comprises applying the composition to the leaves or roots of the plant, or to the soil or growth medium around the plant, optionally wherein said active agent targets a pest of said plant.

7. The method of claim 5, wherein said organism is a human or non-human animal, wherein said administering or applying comprises topically applying said composition to said organism, directly administering said composition to said organism orally or via injection, or introducing said composition into an area where said organism will come into contact with said composition.

8. A method of transfecting a population of cells in vivo or in vitro with nucleic acids, the method comprising:
providing a plurality of peptide capsules according to claim 1, in which said nucleic acids are encapsulated; and
contacting said cells with said plurality of peptide capsules.

9. The method of claim 8, wherein said contacting step comprises: providing said population of cells in cell culture media and incubating said population of cells with said peptide capsules in said cell culture media under transfection conditions.

10. A kit for transfecting a population of cells with nucleic acids, said kit comprising:
a plurality of lyophilized peptide capsules according to claim 1, in which said nucleic acids are encapsulated; and
instructions for reconstituting said lyophilized peptide capsules and incubating said population of cells with said reconstituted peptide capsules.

11. A method of forming a peptide capsule for encapsulating water-soluble active agents, said method comprising:
dispersing or dissolving a plurality of linear peptides in a low-pH buffer system having a pH of less than 5, optionally comprising active agents to be encapsulated, to form a dispersion of peptides and active agent, if present, wherein the peptides each comprise a hydrophobic core of between 4 and 12 hydrophobic amino acids flanked by N- and C-terminal hydrophilic segments each comprising between 3 to 4 hydrophilic amino acids;
incubating the dispersion for a time period of less than 20 minutes, wherein said peptides self-assemble into peptide capsules comprising a membrane having an exterior surface and defining a liquid-receiving interior space, wherein the membrane consists of a plurality of said peptides, wherein said active agent, if present, is encapsulated in said liquid-receiving interior space of said capsule; and
raising the pH of the mixture to a pH of at least about 7.

12. The method of claim 11, wherein the pH of the mixture is raised to said pH of between about 7 and 9 using a neutral or alkaline buffer, preferably wherein said neutral buffer comprises Imadazole-HCl and said alkaline buffer comprises NaOH, or mixtures thereof in water.

13. The method of claim 11, further comprising drying said capsules to a dried or lyophilized powder.

14. The method of claim 13, further comprising rehydrating said dried or lyophilized powder by mixing and suspending said powder in a neutral buffer.

15. A method of forming a peptide capsule for encapsulating water-soluble active agents, said method comprising:
dispersing or dissolving a plurality of linear peptides in an aqueous solution having a pH of from about 2 to about 9, optionally comprising active agents to be encapsulated, to form a dispersion of peptides and active agent, if present, wherein the peptides each comprise a hydrophobic core comprising XLIVIIVIL (SEQ ID NO:47), where X is F or V, flanked by N- and C-terminal hydrophilic segments each comprising between 3 to 4 hydrophilic amino acids;
incubating the dispersion for a time period of less than 20 minutes, wherein said peptides self-assemble into peptide capsules comprising a monolayer membrane having an exterior surface and defining a liquid-receiving interior space, wherein the monolayer membrane consists of a plurality of said peptides, wherein said active agent, if present, is encapsulated in said liquid-receiving interior space of said capsule.

16. The method of claim 15, wherein said hydrophobic core comprises FLIVIIVIL (SEQ ID NO: 47, where X is F).

17. The method of claim 15, wherein said linear peptides comprise KKKFLIVIIVILKKK (SEQ ID NO:56), preferably wherein said membrane consists of said linear peptides.

18. The method of claim 15, wherein said dispersing or dissolving step comprises dispersing or dissolving said plurality of linear peptides at a pH of about 7 in buffer with said active agent.

19. The method of claim 15, further comprising drying said capsules to a dried or lyophilized powder.

20. The method of claim 19, further comprising rehydrating said dried or lyophilized powder by mixing and suspending said powder in a neutral buffer.


SEQUENCE LISTING (57 sequences)

SEQ ID NO: 1 - protein, 5 aa, synthetic construct
  VARIANT 1: note = X is F or V
XLIVI

SEQ ID NO: 2 - protein, 9 aa, synthetic construct
  VARIANT 1: note = X is F or V
XLIVIGSII

SEQ ID NO: 3 - protein, 7 aa, synthetic construct
  VARIANT 1: note = X is F or V
XFFIVIL

SEQ ID NO: 4 - protein, 12 aa, synthetic construct
  VARIANT 1: note = X is F or V
XLIVIGSIIVIL

SEQ ID NO: 5 - protein, 4 aa, synthetic construct
GSII

SEQ ID NO: 6 - protein, 11 aa, synthetic construct
KKKFLIVIKKK

SEQ ID NO: 7 - protein, 15 aa, synthetic construct
KKKFLIVIGSIIKKK

SEQ ID NO: 8 - protein, 5 aa, synthetic construct
  VARIANT 1: note = X is F or V
XLIIV

SEQ ID NO: 9 - protein, 5 aa, synthetic construct
  VARIANT 1: note = X is F or V
XLVII

SEQ ID NO: 10 - protein, 5 aa, synthetic construct
  VARIANT 1: note = X is F or V
XIVIL

SEQ ID NO: 11 - protein, 5 aa, synthetic construct
  VARIANT 1: note = X is F or V
XIIVL

SEQ ID NO: 12 - protein, 5 aa, synthetic construct
  VARIANT 1: note = X is F or V
XILVI

SEQ ID NO: 13 - protein, 5 aa, synthetic construct
  VARIANT 1: note = X is F or V
XVIIL

SEQ ID NO: 14 - protein, 5 aa, synthetic construct
  VARIANT 1: note = X is F or V
XVLII

SEQ ID NO: 15 - protein, 5 aa, synthetic construct
  VARIANT 1: note = X is F or V
XVILI

SEQ ID NO: 16 - protein, 5 aa, synthetic construct
  VARIANT 5: note = X is F or V
LIVIX

SEQ ID NO: 17 - protein, 5 aa, synthetic construct
  VARIANT 4: note = X is F or V
IVIXL

SEQ ID NO: 18 - protein, 5 aa, synthetic construct
  VARIANT 3: note = X is F or V
VIXLI

SEQ ID NO: 19 - protein, 5 aa, synthetic construct
  VARIANT 2: note = X is F or V
IXLIV

SEQ ID NO: 20 - protein, 5 aa, synthetic construct
  VARIANT 5: note = X is F or V
LIIVX

SEQ ID NO: 21 - protein, 5 aa, synthetic construct
IVFLI

SEQ ID NO: 22 - protein, 5 aa, synthetic construct
IIVLF

SEQ ID NO: 23 - protein, 5 aa, synthetic construct
VFLII

SEQ ID NO: 24 - protein, 5 aa, synthetic construct
IIVFL

SEQ ID NO: 25 - protein, 5 aa, synthetic construct
LIVIF

SEQ ID NO: 26 - protein, 5 aa, synthetic construct
IVILF

SEQ ID NO: 27 - protein, 9 aa, synthetic construct
  VARIANT 1: note = X is F or V
XLIVIGSII

SEQ ID NO: 28 - protein, 9 aa, synthetic construct
  VARIANT 1: note = X is F or V
XLIIVGSII

SEQ ID NO: 29 - protein, 9 aa, synthetic construct
  VARIANT 1: note = X is F or V
XLVIIGSII

SEQ ID NO: 30 - protein, 9 aa, synthetic construct
  VARIANT 1: note = X is F or V
XIVILGSII

SEQ ID NO: 31 - protein, 9 aa, synthetic construct
  VARIANT 1: note = X is V or F
XIIVLGSII

SEQ ID NO: 32 - protein, 9 aa, synthetic construct
  VARIANT 1: note = X is V or F
XILVIGSII

SEQ ID NO: 33 - protein, 9 aa, synthetic construct
  VARIANT 1: note = X is F or V
XVIILGSII

SEQ ID NO: 34 - protein, 9 aa, synthetic construct
  VARIANT 1: note = X is F or V
XVLIIGSII

SEQ ID NO: 35 - protein, 9 aa, synthetic construct
  VARIANT 1: note = X is F or V
XVILIGSII

SEQ ID NO: 36 - protein, 7 aa, synthetic construct
  VARIANT 1: note = X is F or V
XFFVIIL

SEQ ID NO: 37 - protein, 7 aa, synthetic construct
  VARIANT 1: note = X is F or V
XFFIIVL

SEQ ID NO: 38 - protein, 12 aa, synthetic construct
  VARIANT 1: note = X is F or V
XLIVIGSIIVIL

SEQ ID NO: 39 - protein, 12 aa, synthetic construct
  VARIANT 1: note = X is F or V
XLIIVGSIIVIL

SEQ ID NO: 40 - protein, 12 aa, synthetic construct
  VARIANT 1: note = X is F or V
XLVIIGSIIVIL

SEQ ID NO: 41 - protein, 12 aa, synthetic construct
  VARIANT 1: note = X is F or V
XIVILGSIIVIL

SEQ ID NO: 42 - protein, 12 aa, synthetic construct
  VARIANT 1: note = X is F or V
XIIVLGSIIVIL

SEQ ID NO: 43 - protein, 12 aa, synthetic construct
  VARIANT 1: note = X is F or V
XILVIGSIIVIL

SEQ ID NO: 44 - protein, 12 aa, synthetic construct
  VARIANT 1: note = X is F or V
XVIILGSIIVIL

SEQ ID NO: 45 - protein, 12 aa, synthetic construct
  VARIANT 1: note = X is F or V
XVLIIGSIIVIL

SEQ ID NO: 46 - protein, 12 aa, synthetic construct
  VARIANT 1: note = X is F or V
XVILIGSIIVIL

SEQ ID NO: 47 - protein, 9 aa, synthetic construct
  VARIANT 1: note = X is F or V
XLIVIIVIL

SEQ ID NO: 48 - protein, 9 aa, synthetic construct
  VARIANT 1: note = X is F or V
XLIIVIVIL

SEQ ID NO: 49 - protein, 9 aa, synthetic construct
  VARIANT 1: note = X is F or V
XLVIIIVIL

SEQ ID NO: 50 - protein, 9 aa, synthetic construct
  VARIANT 1: note = X is F or V
XIVILIVIL

SEQ ID NO: 51 - protein, 9 aa, synthetic construct
  VARIANT 1: note = X is F or V
XIIVLIVIL

SEQ ID NO: 52 - protein, 9 aa, synthetic construct
  VARIANT 1: note = X is F or V
XILVIIVIL

SEQ ID NO: 53 - protein, 9 aa, synthetic construct
  VARIANT 1: note = X is F or V
XVIILIVIL

SEQ ID NO: 54 - protein, 9 aa, synthetic construct
  VARIANT 1: note = X is F or V
XVLIIIVIL

SEQ ID NO: 55 - protein, 15 aa, synthetic construct
  VARIANT 4: note = X is F or V
KKKXLIVIIVILKKK

SEQ ID NO: 56 - protein, 15 aa, synthetic construct
KKKFLIVIIVILKKK

SEQ ID NO: 57 - protein, 4 aa, synthetic construct
IVIL